(19) United States
(12) Patent Application Publication
(10) Pub. No.: US 2015/0038803 A1
(43) Pub. Date: Feb. 5, 2015

(54) System and Method for Evaluating Concussion Injuries

(71) Applicant: Motion Intelligence LLC, Ithaca, NY (US)

(72) Inventors: Richard A. Uhlig, Ithaca, NY (US); Andrew Brindle, Clay, NY (US)

(21) Appl. No.: 14/327,906

(22) Filed: Jul. 10, 2014

Related U.S. Application Data
(60) Provisional application No. 61/861,715, filed on Aug. 2, 2013.
(60) Provisional application No. 61/991,743, filed on May 12, 2014.
(60) Provisional application No. 62/011,761, filed on Jun. 13, 2014.

(51) Int. Cl. A61B 5/00; G06F 19/00; A61B 5/16

(52) U.S. Cl. CPC A61B 5/4064; A61B 5/16; A61B 5/4023; G06F 19/3487
USPC 600/301

(57) ABSTRACT

A portable and cost-effective method and system for evaluating a subject's concussion symptoms, testing their cognitive and motor abilities, and evaluating those abilities when performed concurrently.

### REFERENCE TO RELATED APPLICATIONS

This application claims one or more inventions which were disclosed in the following Provisional Applications: No. 61/861,715, filed Aug. 2, 2013, entitled “DUAL-TASK EVALUATION SYSTEM”; No. 61/991,743, filed May 12, 2014, entitled “System and Method for Evaluating Postural Stability”; and No. 62/011,761, filed Jun. 13, 2014, entitled “System and Method for Evaluating Concussion Injuries”. The benefit under 35 USC §119(e) of the aforementioned United States provisional applications is hereby claimed, and the aforementioned applications are hereby incorporated herein by reference.

### BACKGROUND OF THE INVENTION

1. Field of the Invention

The invention relates to the periodic assessment and quantification of certain signs and symptoms associated with concussion injuries in humans. Specifically, the invention is a portable method and system for evaluating a subject's concussion symptoms, testing their cognitive and motor abilities, and evaluating those abilities when performed concurrently; results are evaluated on a stand-alone basis and relative to prior testing.

2. Description of Related Art

A concussion injury is often identified through the self-reporting of various somatic, cognitive, or neurobehavioral symptoms; traditionally, injury recovery is marked by the abatement of those symptoms. However, research has demonstrated that certain cognitive tests and certain motor tests (including measurements of postural stability) are sensitive to concussion injuries; further research indicates that dual-task testing (the combination of cognitive testing while the subject is engaged in a challenging motor task) may identify persistent or lingering effects of brain injuries after the abatement of symptoms and not otherwise perceivable through stand-alone cognitive or motor testing. Recognition of on-going deficits may reduce the occurrence of subsequent brain injuries and limit further damage from premature return-to-play or return-to-duty decision.

While the collection of symptoms and cognitive testing can be administered in nearly any venue, accurately detecting changes in a person's postural stability can be challenging outside of a clinical research environment and/or on a real-time basis.

The collection of self-reported symptoms was guided by prior art, namely:
Piland S G, M. R. (2003). Evidence for the Factorial and Construct Validity of a Self-Report Concussion Symptoms Scale. Journal of Athletic Training, 38(2), 104-112 .
Piland S G, M. R. (2006). Structural Validity of a Self-Reported Concussion-Related Symptom Scale. Medicine & Science in Sports & Exercise, 38(1), 27-32.
Randolph C, M. S. (2009). Concussion Symptom Inventory: an empirically derived scale for monitoring resolution of symptoms following sport-related concussion. Archives of Clinical Neuropsychology, 1-11. doi:10.1093/arclin/acp025

The use of cognitive tests in concussion evaluations was informed by:
Broglio S P, F. M. (2007). Test-Retest Reliability of Computerized Concussion Assessment Programs. Journal of Athletic Training, 42(4), 509-514.
Galetta M S, G. K. (2013). Saccades and memory: Baseline associations of the King-Devick and SCAT2 SAC tests in professional ice hockey players. Journal of the Neurological Sciences, 328, 28-31.
Guskiewicz K M, R. B. (1997). Alternative approaches to the assessment of mild head injuries in athletes. Medicine & Science in Sports & Exercise, 27(7 Supplement), 213-221.
Guskiewicz K M, R. S. (2001). Postural Stability and Neuropsychological Deficits After Concussion in Collegiate Athletes. Journal of Athletic Training, 36(3), 263-273.
The ImPACT® Test (Immediate Post Concussion Assessment Cognitive Testing), described in a web page at http://www.impacttest.com, at least as early as 2001.
The Concussion Resolution Index, described in a web page at http://www.headminder.com/site/cri/home.html, at least as early as 2001.

The use of balance or other motor tasks in concussion evaluations was informed by:
The Balance Error Scoring System (BESS), University of North Carolina Sports Medicine Research Laboratory, June 2009
Cripps A, L. S. (2013). The Value of Balance-Assessment Measurements in Identifying and Monitoring Acute Postural Instability Among Concussed Athletes. Journal of Sport Rehabilitation, 22, 67-71.
Fait P, M. B. (2009). Alterations to locomotor navigation in a complex environment at 7 and 30 days following a concussion in an elite athlete. Brain Injury, 1-8.
Wilkins J C, V. T. (2004). Performance on the Balance Error Scoring System Decreases After Fatigue. Journal of Athletic Training, 39(2), 156-161.

The combined use of balance and cognitive testing in concussion evaluations was informed by:
Concussion in Sport Group—Sport Concussion Assessment Tool 3 (2013).

The use of dual-task testing in concussion evaluations was informed by:
Broglio S P, T. P. (2005). Balance Performance with a Cognitive Task: A Dual-Task Testing Paradigm. Medicine 7 Science in Sports & Exerices, 689-695.
Catena R D, v. D. (2007). Altered Balance Control following Concussion is Better Detected with and Attention Test During Gait. Gait and Posture, 25(3), 406-411.
Catena R D, v. D. (2011). The Effects of Attention Capacity on Dynamic Balance Control Following Concussion. Journal of Neroengineering and Rehabilitation, 8, 8.
Howell D R, O. L. (2013). Dual-Task Effect on Gait Balance Control in Adolescents With Concussion. Archives of Physical Medicine and Rehabilitation, 1513-1520.
Register-Mihalik J K, L. A. (2013 Nov. 17). Are Divided Attention Tasks Useful in the Assessment and Management of Sport-Related Concussion. Neuropsychol Rev, 1-14.
Resch J E, M. B. (2011, April). Balance Performance with a Cognitive Task: A Continuation of the Dual-Task Testing Paradigm. Journal of Athletic Training, 46(2), 170-175.
Teel E F, R.-M. J. (2013). Balance and cognitive performance during a dual-task: Preliminary implications for use in concussion assessment. Journal of Science and Medicine in Sport, 16, 190-194.

The use of accelerometer-based tools for the assessment of postural stability was informed by:
U.S. Pat. No. 8,529,448, “Computerized Systems and Methods For Stability—Theoretic Prediction and Prevention of Falls”, McNair, issued Sep. 10, 2013
APDM wearable inertial monitors manufactured by APDM, Inc., of Portland, Oreg.
Furman G R, L. C. (2013). Comparison of the Balance Accelerometer Measure and Balance Error Scoring System in Adolescent Concussions in Sports. The American Journal of Sports Medicine, 41(6), 1404-1410.
Mancini M, S. C. (2012). ISway: a Sensitive, Valid and Reliable Measure of Postural Control. Journal of NeuroEngineering and Rehabilitation. 9(59), 1-8.
Sway Medical LLC. (2013 Jul. 23). Smartphone Sensitivity in Object Balance Testing.

### SUMMARY OF THE INVENTION

A concussion injury is often identified through the self-reporting of various somatic, cognitive, or neurobehavioral symptoms; traditionally, injury recovery is marked by the abatement of those symptoms. However, research has demonstrated that certain cognitive tests and certain motor tests (including measurements of postural stability) are sensitive to concussion injuries; further research indicates that dual-task testing (the combination of cognitive testing while the subject is engaged in a challenging motor task) may identify persistent or lingering effects of brain injuries after the abatement of symptoms and not otherwise perceivable through stand-alone cognitive or motor testing. Recognition of on-going deficits may reduce the occurrence of subsequent brain injuries and limit further damage from premature return-to-play or return-to-duty decision.

While the collection of symptoms and cognitive testing can be administered in nearly any venue, accurately detecting changes in a person's postural stability can be challenging outside of a clinical research environment and/or on a real-time basis. The invention is a portable and cost-effective method and system for evaluating a subject's concussion symptoms, testing their cognitive and motor abilities, and evaluating those abilities when performed concurrently; results are evaluated on a stand-alone basis and relative to prior testing.

The invention provides a portable and cost-effective method and apparatus for the measurement and processing of motion data collected at the subject's approximate center of mass such that physiologically meaningful information is obtained about a subject's postural stability. The method and apparatus includes a means of measuring a subject's three dimensional motion when: the subject is standing quietly with feet together and eyes open on a firm surface; the subject's visual input is removed; the subject stands on an uncertain surface; and, the subject stands in a physically challenging stance. Physiologically meaningful information about a subject's postural stability and balance is determined using mathematical techniques and statistical analysis to manipulate the subject's inertial motion data as gathered by a purpose-built inertial measurement device worn by the subject.

The invention provides a portable and cost-effective method and apparatus for the administration and scoring of certain dual-task tests (such tests involving the combination of one or more cognitive tests while the subject is engaged in a challenging postural stability task).

The invention provides a method and system for the real-time evaluation of (i) a subject's current concussion symptoms, cognitive scores, postural stability scores, and dual-task scores, (ii) any changes from prior testing, and (iii) current test performance versus peer-group statistics.

### BRIEF DESCRIPTION OF THE DRAWING

FIG. 1 is a representation of the postural stability analysis system using a wired device.

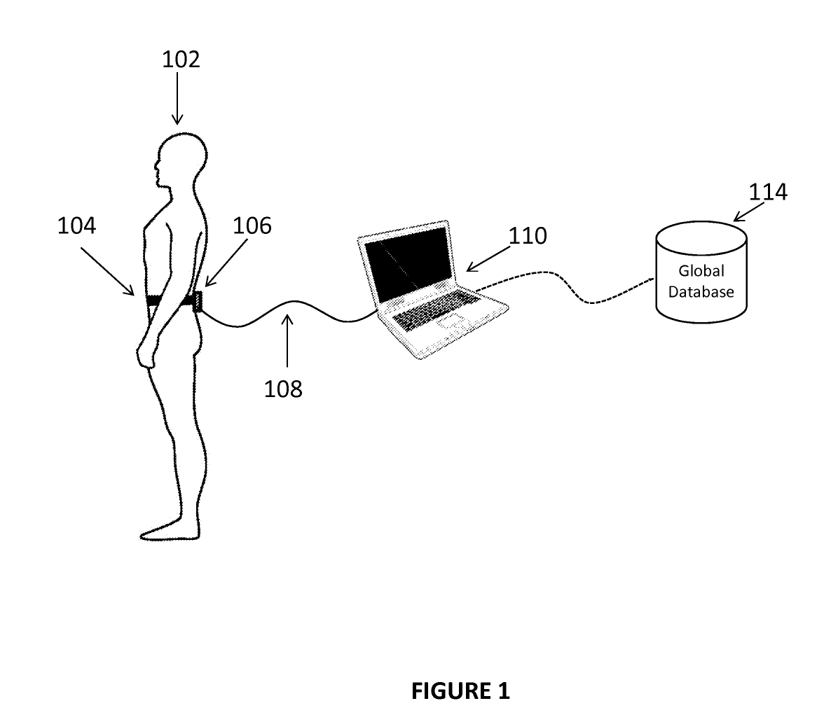

FIG. 2 is a representation of the postural stability analysis system using a wired device with a subject standing on an uncertain (foam) surface.

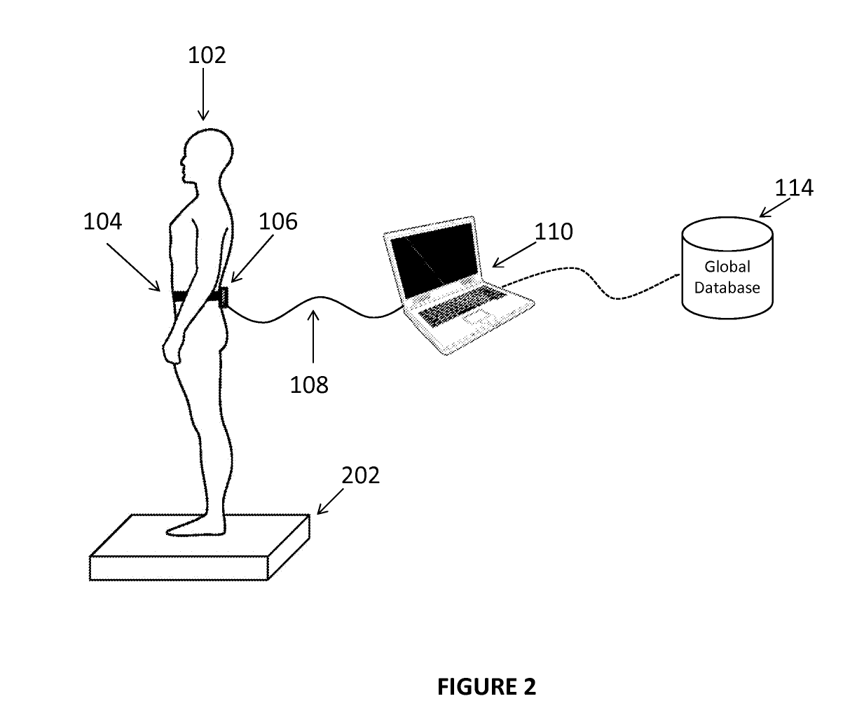

FIG. 3 is a representation of the postural stability analysis system using a wireless device.

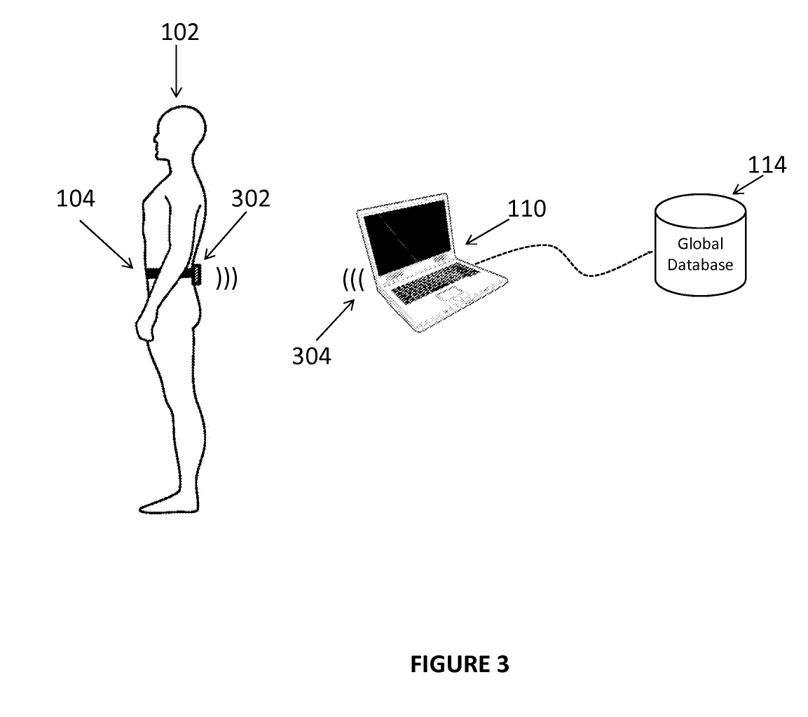

FIG. 4 is a representation of the postural stability analysis system using a wireless device with a subject standing on an uncertain (foam) surface.

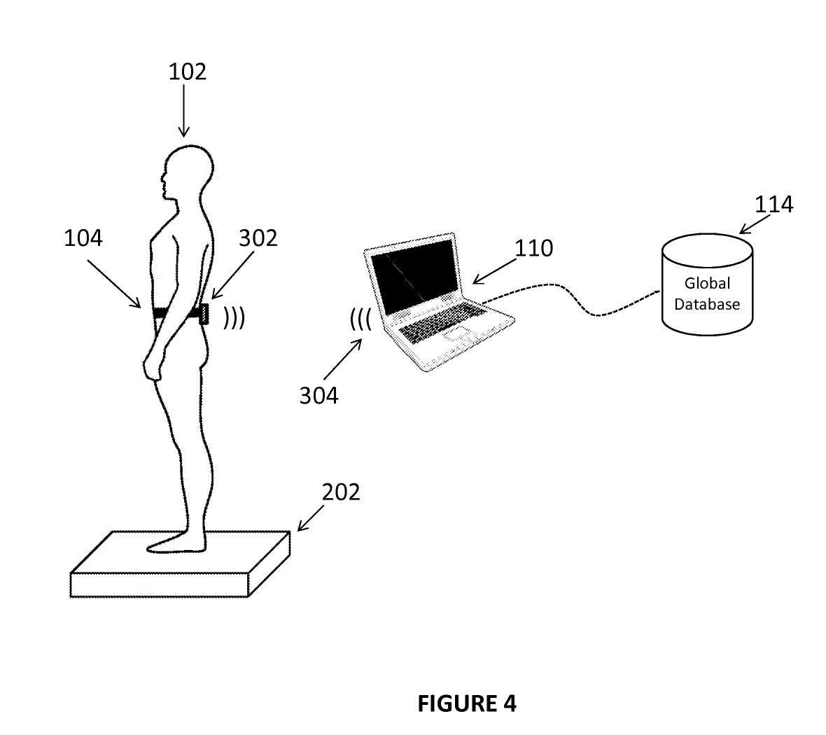

FIG. 5 is a block diagram identifying the critical components of the wired device.

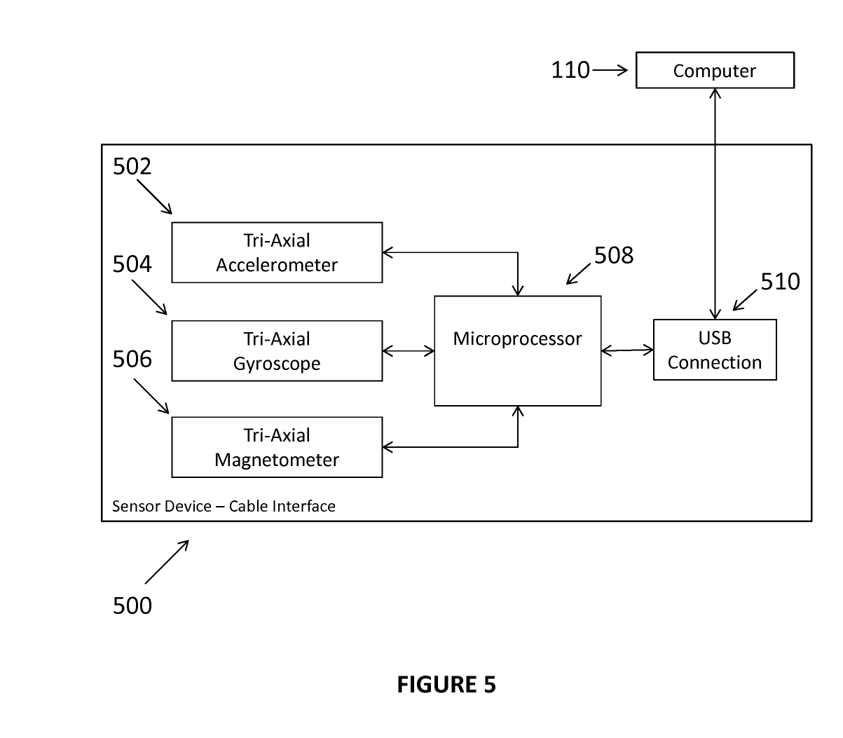

FIG. 6 is a block diagram identifying the critical components of the wireless device.

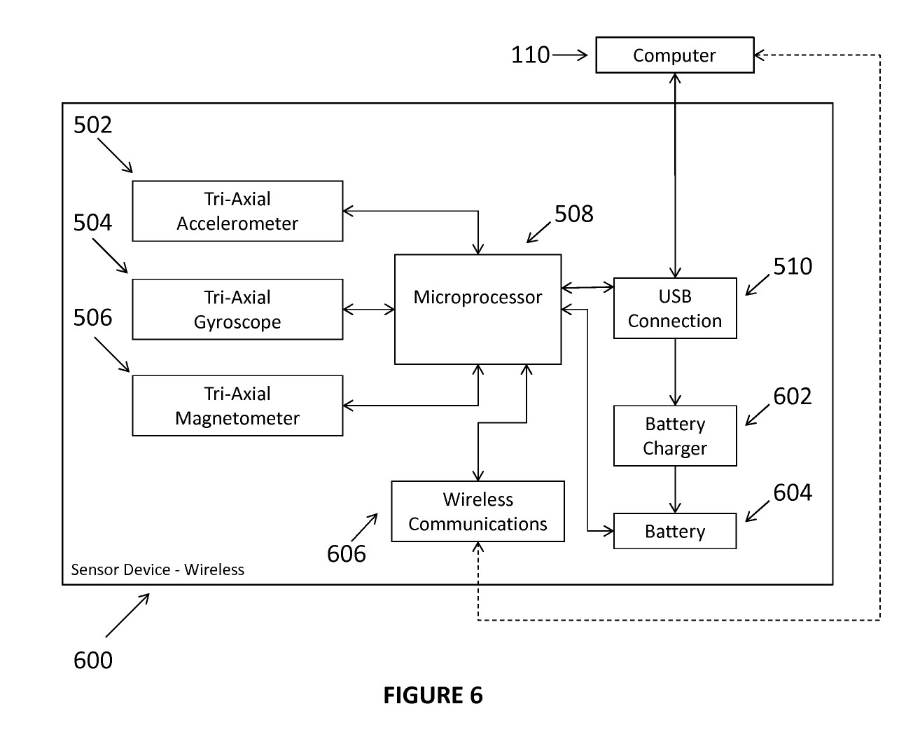

FIG. 7 is a block diagram representing the major functions performed on the device microprocessor.

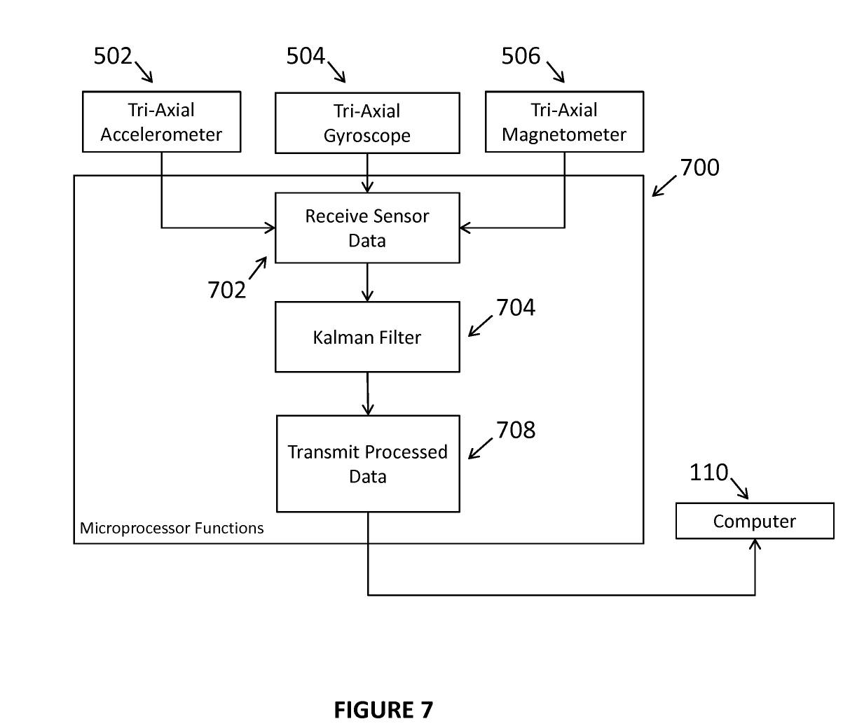

FIG. 8 is a schematic of the postural stability testing methodology.

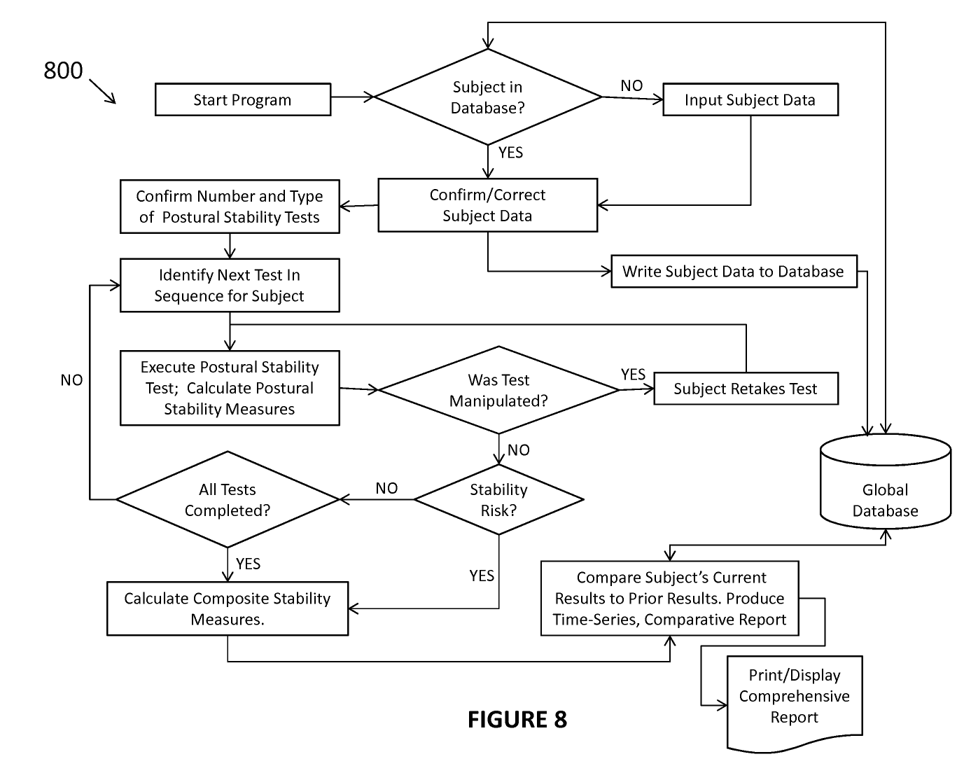

FIG. 9 is a representation of the four postural stability tasks performed on a firm surface.

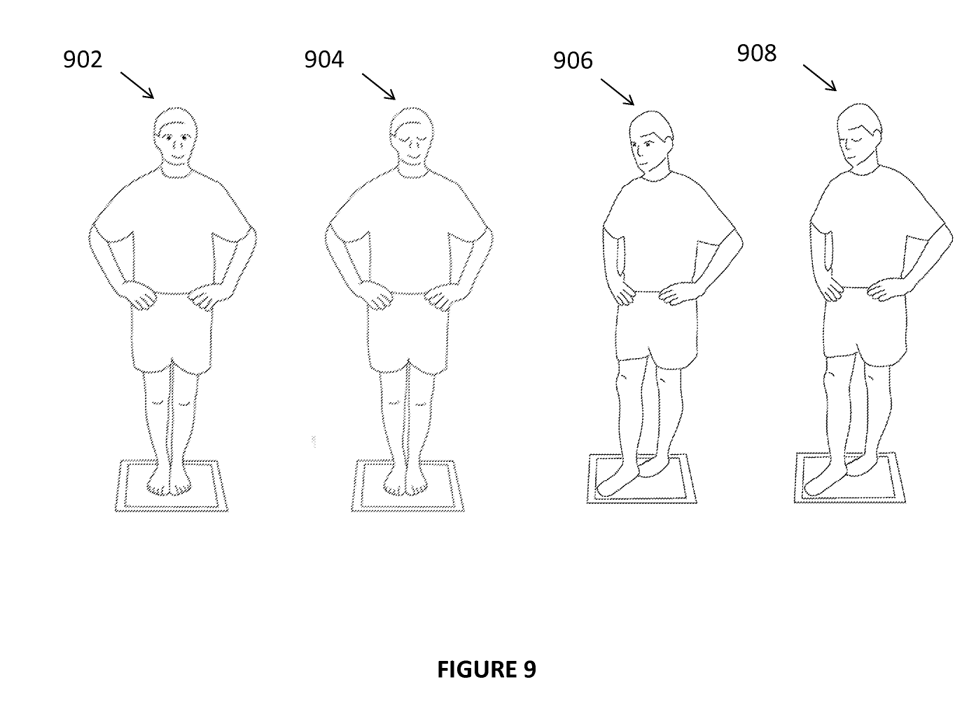

FIG. 10 is a representation of the four postural stability tasks performed on a foam surface.

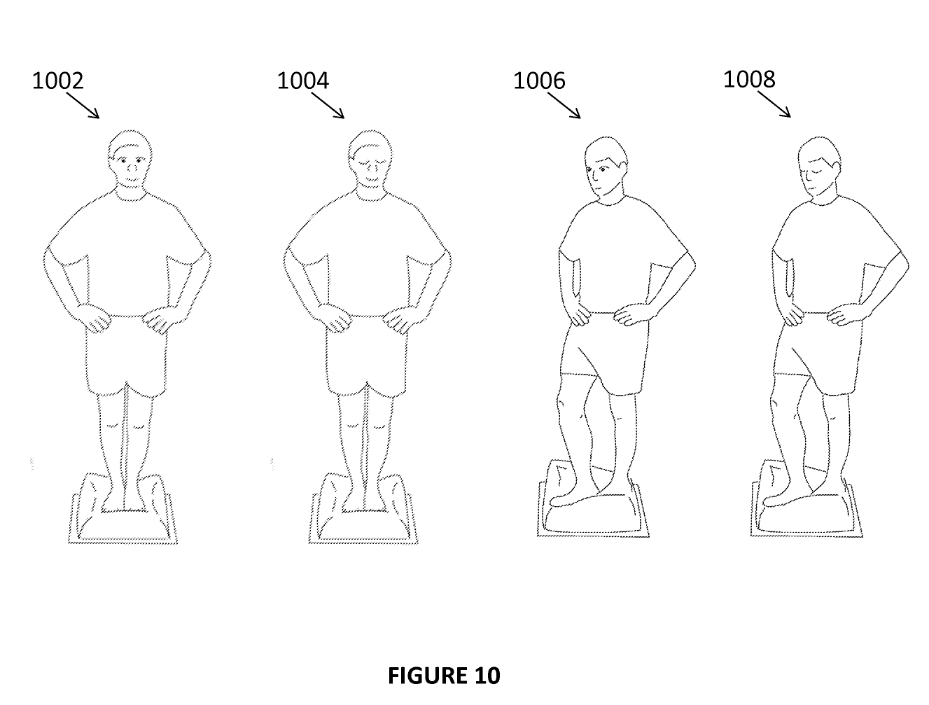

FIG. 11 is a representation of a postural stability analysis report.

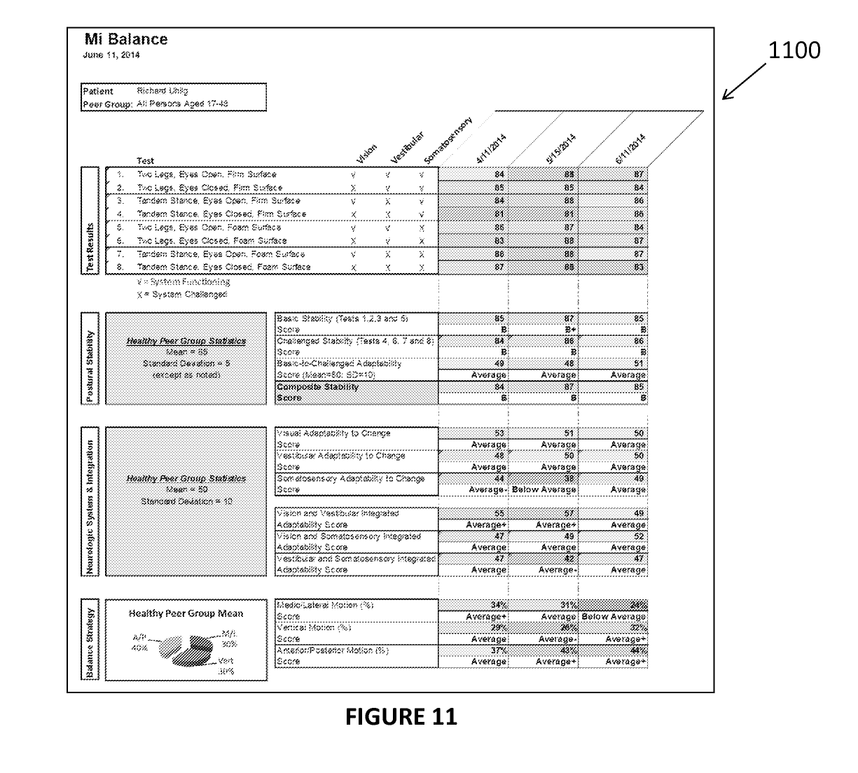

FIG. 12 is a representation of the purpose-built IMU protective enclosure.

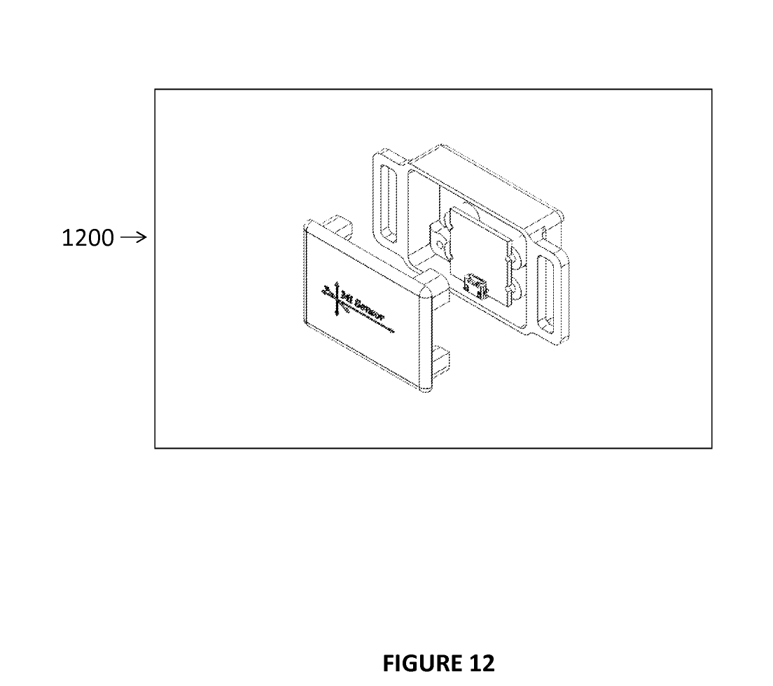

FIG. 13 is a block diagram identifying the relationship between various concussion symptoms.

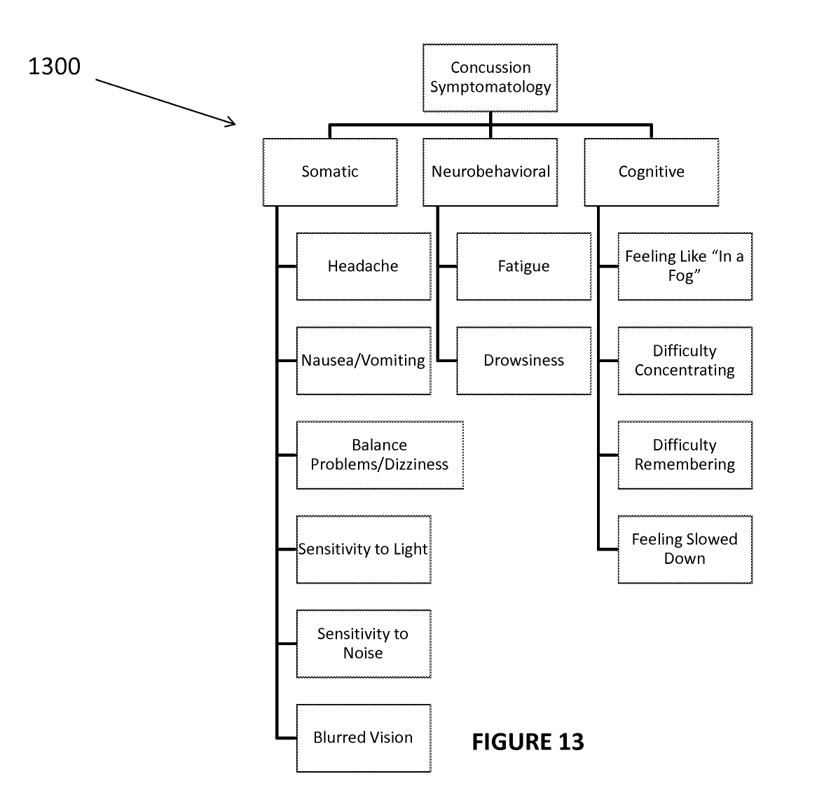

FIG. 14 is a graded symptom checklist.

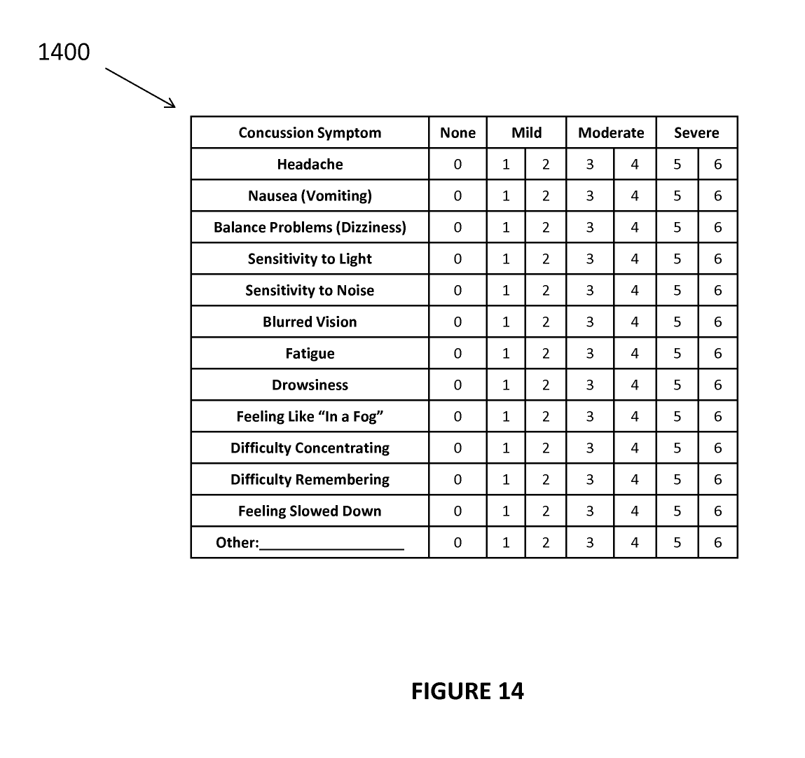

FIG. 15 is a block diagram identifying the proscribed test sequence.

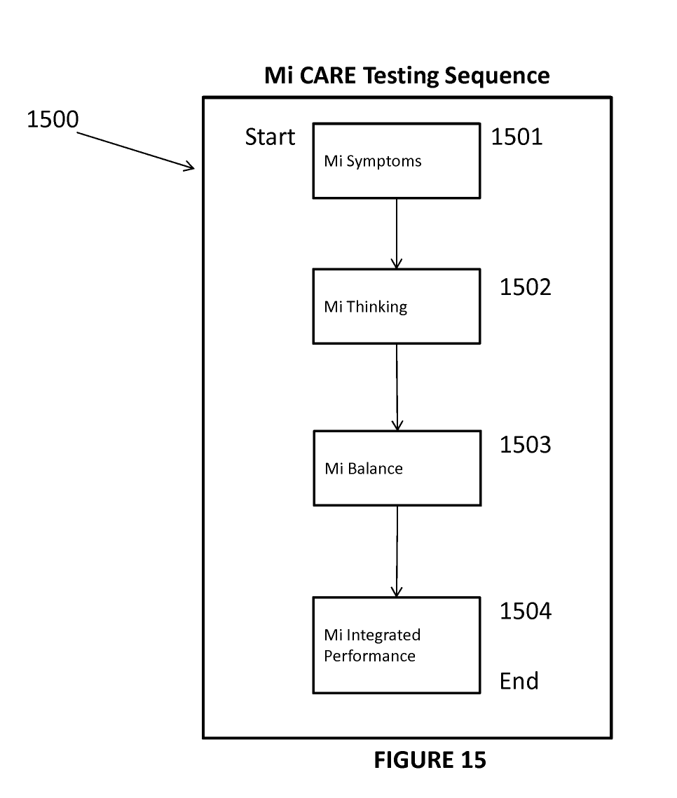

FIG. 16 is a representation of the symptom collection and cognitive testing system.

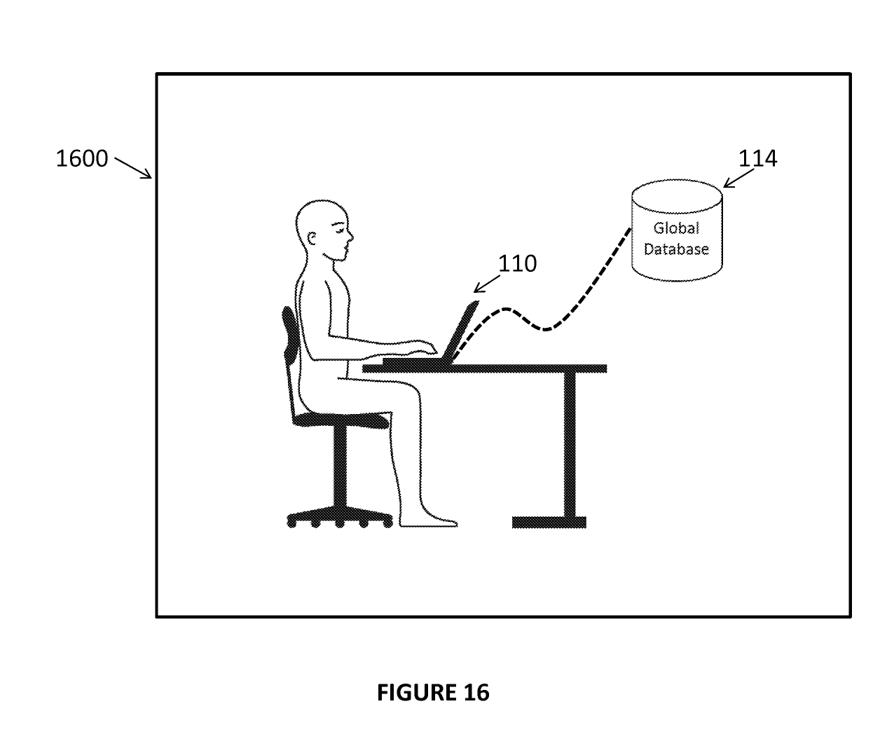

FIG. 17 is a representation of a challenging postural stability task performed while a person is taking a computerized cognitive test (dual-task testing).

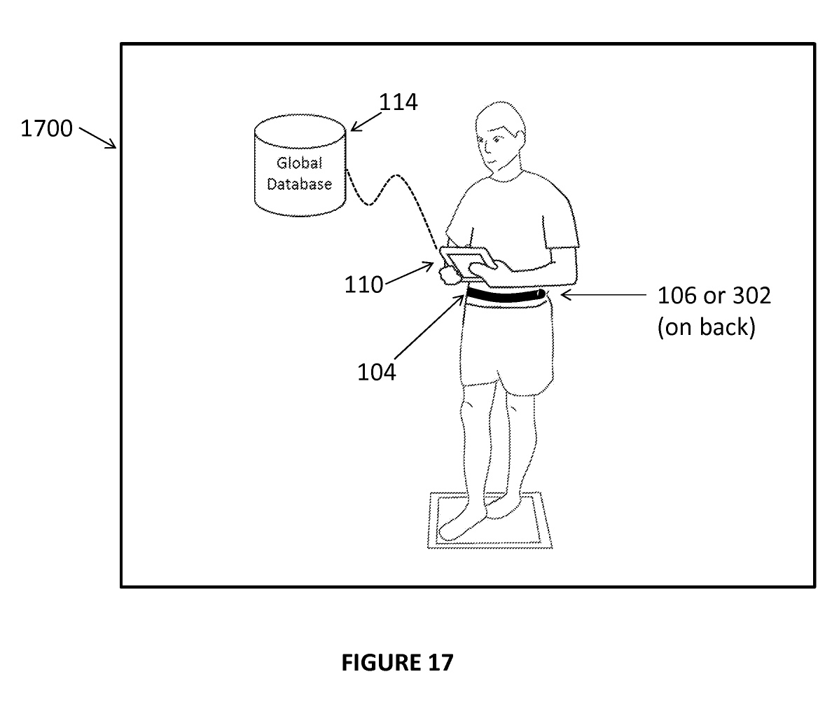

FIG. 18 is a diagram representing the components of the Mi CARE system.

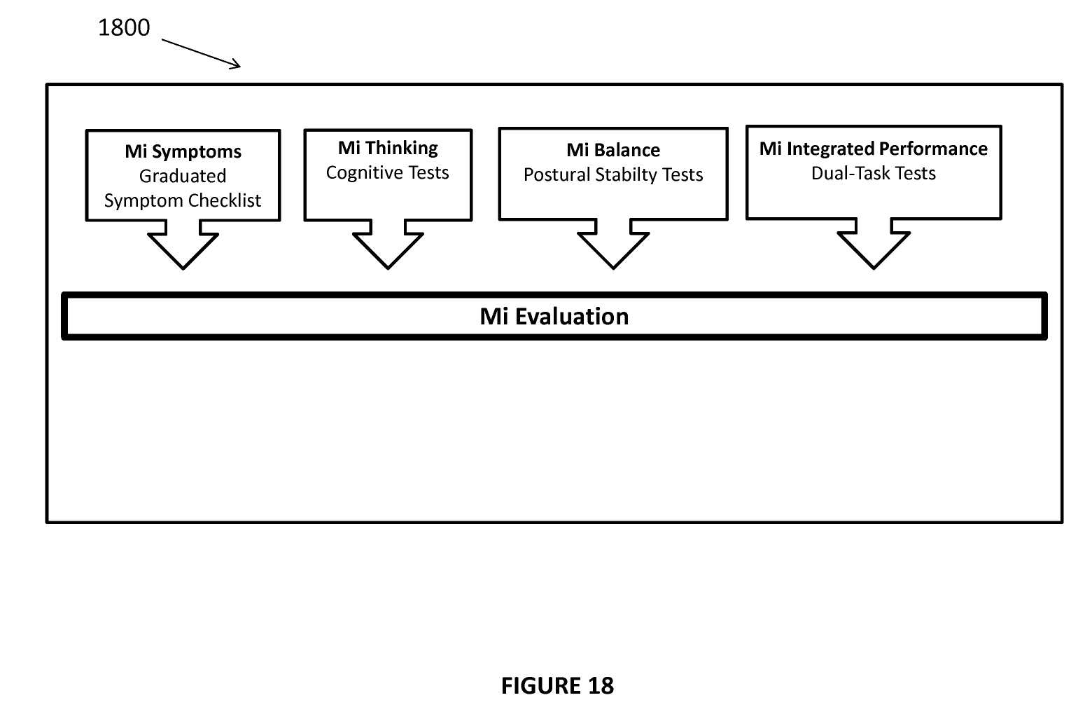

FIG. 19 is a representation of a Mi Evaluation summary report.

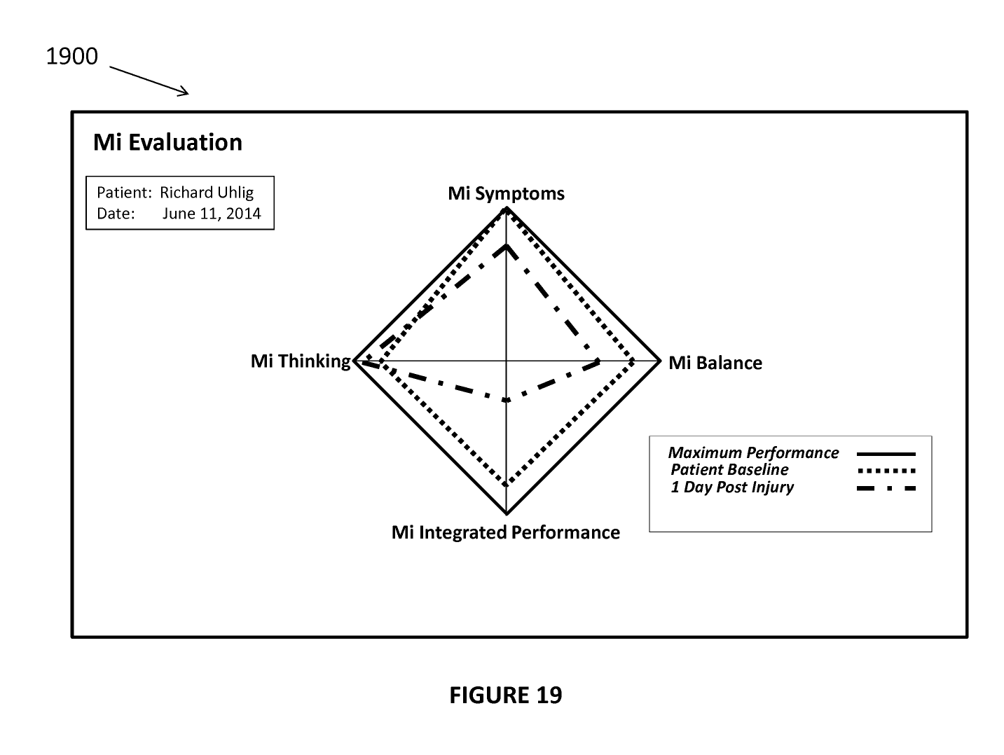

FIG. 20 is a representation of a concussion symptoms analysis report.

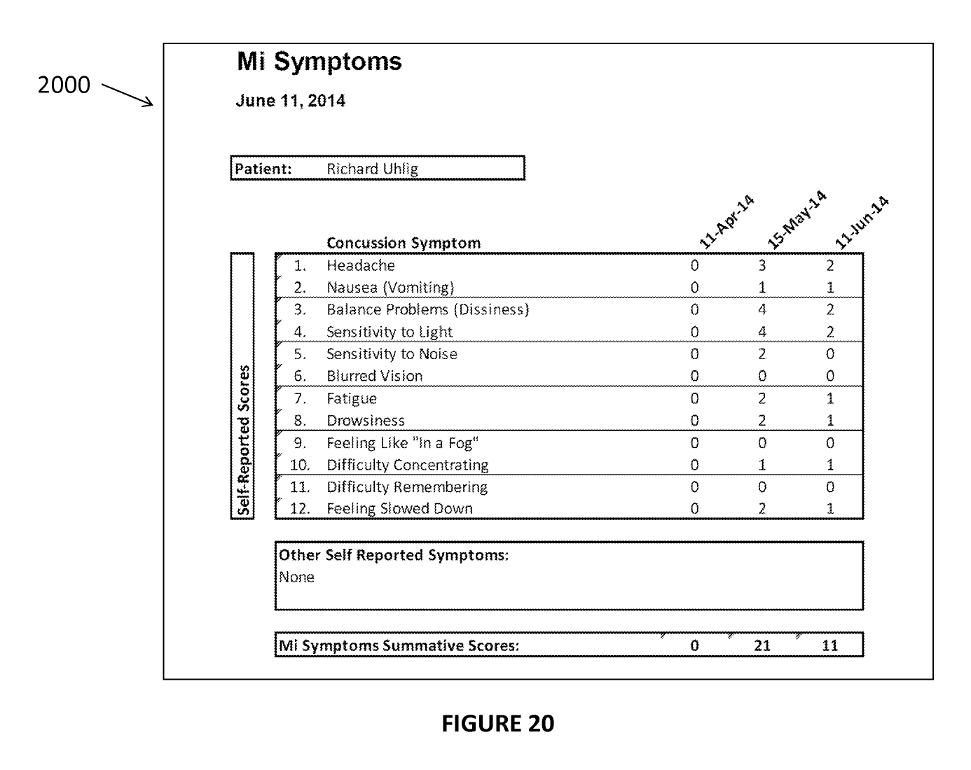

FIG. 21 is a representation of a cognitive testing analysis report.

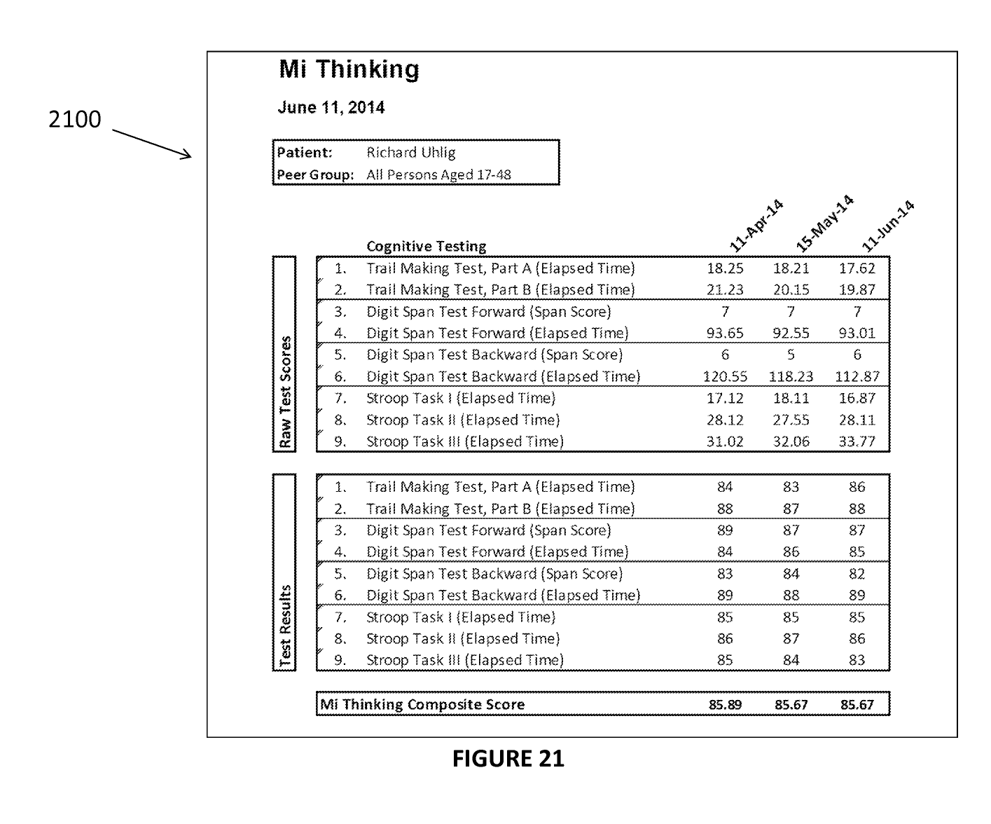

FIG. 22 is a representation of an integrated cognitive and postural stability testing analysis report.

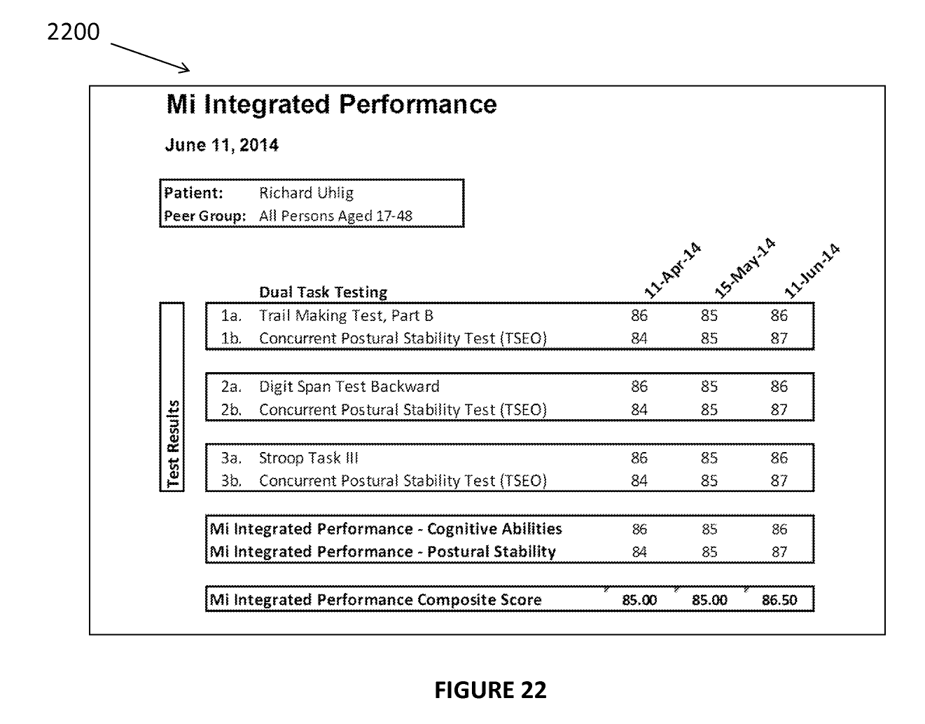

### DETAILED DESCRIPTION OF THE INVENTION

The principal components of the Motion Intelligence Concussion Assessment and Recovery Evaluation System (the “Mi CARE System”) (1800) include: a system and method for the collection of self-reported symptoms (“Mi Symptoms”); a system and method for administering and scoring one or more cognitive tests (“Mi Thinking”); a system and method for administering and scoring certain postural stability tests (“Mi Balance”); a system and method for administering and scoring certain dual-task tests (“Mi Integrated Performance”); for each of Mi Symptoms, Mi Thinking, Mi Balance and Mi Integrated Performance, a system and method to retain elements of a patient's symptoms and test history; and a system and method of reporting test results which provides physicians or other health-care providers with concise and objective data to facilitate patient diagnosis (“Mi Evaluation”).

### Testing Sequence

The Mi Care System requires a prescribed testing sequence (1500), specifically:
First—the collection of self-reported symptoms through Mi Symptoms (1501);
Second—the administration and scoring of one or more cognitive test through Mi Thinking (1502);
Third—the administration and scoring of certain postural stability tests through Mi Balance (1503); and
Fourth—the administration and scoring of certain dual-task tests through Mi Integrated Performance (1504).

### Mi Symptoms

The Mi Symptoms component of the invention systematically collects and stores symptom data from potentially concussed or recovering persons using either a computer-based program or otherwise. In the preferred embodiment of the invention, Mi Symptoms employs a graded symptom checklist using a 7-point Likert scale and 12 self-reported concussion symptoms (1400) that can be explained by three underlying latent variables, namely somatic symptoms, neurobehavioral symptoms, and cognitive symptoms (1300).

In the preferred embodiment of the invention, the collection of symptoms data will occur electronically on a computer or tablet while the subject is seated comfortably at a desk or table (1600); a central database of collected data and processed information (the “Global Database”) (114) will be accessible by the computer (110) for the retention of symptoms data and prospective comparative analysis.

In the preferred embodiment, a “Mi Symptoms Summative Score” is calculated as the summation of the self-reported symptoms, with each of the 12 symptoms being graded on a scale of zero to 6. The summative score in this embodiment can range from a minimum of zero to a maximum of 72. It will be understood that other numeric scoring values are possible, as well as other numbers of symptoms. It will also be understood that if desired, the scale can be inverted for graphic purposes by subtracting the summation from the possible maximum, so that a total of zero would represent maximum symptoms and 72 (in the example above) would represent no symptoms.

Following the collection of data as described above, an “Mi Symptoms” concussion symptoms analysis report is generated relative to the subject (2000). In the preferred embodiment, the Mi Symptoms concussion symptoms analysis report contains the self-reported scores and the Mi Symptoms Summative Score for the current testing date and each previous testing date.

### Mi Thinking

The Mi Thinking component of the invention is a system used to evaluate elements of a person's cognitive abilities and changes in those cognitive abilities over time. The system administers and scores one or more neuropsychological tests; all such tests are proprietary derivations of one or more similar tests for which, in clinical evaluations, human subjects have exhibited lowered neuropsychological performance following concussion injuries. Examples of such tests include: the Trail-Making Test, Parts A & B; the Digit Span Test, Forward and Backward (from the Wechsler Adult Intelligence Scale); and the Stroop Task.

In the preferred embodiment, the administration and scoring of the cognitive tests will be conducted electronically through subject interaction with software resident on a computer while the subject is seated comfortably at a desk or table (1600); software resident on the computer (110) will calculate the person's cognitive test score(s); cognitive test data will be transmitted to the Global Database (114); certain elements of the Global Database will be accessible by the computer for comparative analysis. The objective methods used to score the test(s) will be dependent on the nature of the test(s), but will generally include one or more timed tasks and a may include other objective criteria. In cases where a person is periodically retested, a pre-injury Mi Thinking “baseline” is calculated as the subject's best test score (i.e. in the case of a test scoring rubric which measures elapsed time, the shortest time to complete the test will be the subject's pre-injury baseline score).

For each cognitive test associated with a specific subject (person), we calculate a score relative to a selected cohort or peer group:

From the Global Database of collected information, a specific peer group may be formed by sorting the database by one or more characteristics collected for each subject (such as age, gender, height, weight, health factor, etc.); for the selected peer group, the mean (“MEAN”) and standard deviation (“SD”) values are calculated for each of the test scoring criteria (such as elapsed time) for each test.

For each test scoring criteria, the peer group MEAN, +/−1 SD and +/−2SD will each be assigned an ordinal value. In the preferred embodiment, the peer groups will be selected from healthy subjects and the MEAN will be assigned an ordinal value of 85; +1 SD and −1 SD will be assigned values of 90 and 80, respectively; +2 SD and −2 SD will be assigned values of 95 and 75, respectively; no score can exceed 100 nor be less than zero.

Based on the selected peer group curve, an ordinal value is assigned to each of the scoring criteria values for each cognitive test associated with a specific subject. Each such ordinal value will also be assigned an interval value. In the preferred embodiment, ordinal values of zero through 59 will have an interval value of “F”; ordinal values of 60 through 69 will have an interval value of “D”; ordinal values of 70 through 72 will have and interval value of “C−”; ordinal values of 73 through 76 will have an interval value of “C”; ordinal values of 77 through 79 will have and interval value of “C+”; ordinal values of 80 through 82 will have and interval value of “B−”; ordinal values of 83 through 86 will have an interval value of “B”; ordinal values of 87 through 89 will have and interval value of “B+”; ordinal values of 90 through 92 will have and interval value of “A−”; ordinal values of 93 through 96 will have an interval value of “A”; ordinal values of 97 through 100 will have and interval value of “A+”.

For each subject, we calculate a composite score relative to a selected cohort or peer group:

Using the per-test ordinal and interval values assigned above, a weighted average “Mi Thinking Composite Score” is calculated including the scores from all administered Mi Thinking tests. In the preferred embodiment, the weighting of each test is equal.

Following the calculations described above, a “Mi Thinking” cognitive abilities analysis report is generated relative to the subject. In the preferred embodiment, the Mi Thinking cognitive abilities analysis report contains the Mi Thinking Composite Score and the ordinal and/or interval scores for each of the administered tests for the current testing session and for each of the prior testing sessions (2100)

### Mi Balance

The Mi Balance component of the invention is a system used to evaluate a person's postural stability and changes in postural stability over time. The system measures and records a plurality of inertial motion data while the subject (a person) (102) executes a plurality of physical tasks. The inertial motion data are processed by a connected mobile computer for meaningful analysis and use by trained personnel.

The system utilizes one or more inexpensive, non-invasive, portable and wearable inertial motion sensing and reporting units (each an “IMU”) encapsulated within a purpose-built protective enclosure (106 for the wired IMU; 302 for the wireless IMU), an integrated fitment device worn by the subject (104), a computer (110) connected either wirelessly (304) or via cable interface (108) to the IMU(s), software used to calculate parameters associated with a person's postural stability, a central database of collected data and processed information (the Global Database) (114)) accessible by the computer (110), and, for certain tests, a foam pad (202).

In one embodiment, the IMU includes a tri-axial accelerometer (502), tri-axial gyroscope (504), tri-axial magnetometer (506), an embedded microprocessor (508) and a USB port (510) (collectively, the “Wired-IMU” (500)). The Wired-IMU is connected to a mobile computer via cable interface (108).

In another embodiment, the IMU also includes a wireless communications module (606), a battery (604) and a battery charger (602) (collectively, the “Wireless-IMU” (600)). The Wireless-IMU is connected to a mobile computer through wireless communications such as Bluetooth or other similar technology.

The IMU is housed in a purpose-build protective enclosure (1202) and attached to a purpose-built fitment device (1204); in the preferred embodiment, the purpose-built fitment device is a belt that can be adjusted to fit a most subject waist sizes. In the preferred embodiment of the methodology, the IMU, which is housed in a protective enclosure, is to be securely attached to the subject using the fitment device, near the subject's center of mass (in the center of the lower back, approximately at the 5^{th} lumbar vertebrae).

The IMU samples certain data, preferably at over 1,000 Hz (702), before application of a Kalman filter (704); sensor data is available in excess of 240 Hz post-filter and includes: a timestamp, Quaternion X (“Q_{X}”), Quaternion Y (“Q_{Y}”), Quaternion Z (“Q_{Z}”), Quaternion W (“Q_{W}”), Acceleration X (“A_{X}”), Acceleration Y (“A_{Y}”), Acceleration Z (“A_{Z}”), Gyroscope X (“G_{X}”), Gyroscope Y (“G_{Y}”), Gyroscope Z (“G_{Z}”), Compass X (“C_{X}”), Compass Y (“C_{Y}”), Compass Z (“C_{Z}”) (collectively, the “Processed Data”). The Processed Data is then transmitted (708) to the computer.

For certain calculations, A_{X}, A_{Y} and A_{Z} are subject to additional filtering on the computer, resulting in A_{XF}, A_{YF} and A_{ZF}; in the preferred embodiment, this additional filtering consists of a first-order, low-pass Butterworth filter at 20 Hz.

Certain biometric and identifying data associated with the test subjects will be collected and stored in the Global Database.

While wearing an IMU connected to a mobile computer, subjects will be asked to perform certain tasks which test their postural stability under varying conditions and in accordance with a specific sequence of events; data collected will be stored in the Global Database; and a comprehensive report will be provided to the subject and/or the test administrator (collectively, the “Testing Methodology”) (800).

In the preferred embodiment of the Testing Methodology, IMU data is collected while a subject performs eight motor tasks, each task having a specified duration. In the preferred embodiment, the time duration for each motor task is 30 seconds. In other embodiments of the testing methodology, only a subset of these eight motor tasks are performed by the subject; in yet other embodiments of the testing methodology, the IMU may collect data while the subject is walking, running or performing some other motor task. In the preferred embodiment, the eight motor tasks include:

a) Two Legs, Eyes Open, Firm Surface (“TLEO”) (902);

b) Two Legs, Eyes Closed, Firm Surface (“TLEC”) (904);

c) Tandem Stance, Eyes Open, Firm Surface (“TSEO”) (906);

d) Tandem Stance, Eyes Closed, Firm Surface (“TSEC”) (908);

e) Two Legs, Eyes Open, Foam Pad (“TLEOFP”) (1002);

f) Two Legs, Eyes Closed, Foam Pad (“TLECFP”) (1004);

g) Tandem Stance, Eyes Open, Foam Pad (“TSEOFP”) (1006); and

h) Tandem Stance, Eyes Closed, Foam Pad (“TSECFP”) (1008).

In the preferred embodiment, the foam pad (202) is an Airex Balance Pad.

Prior to performing each motor task, a “tare function” is executed whereby the starting X, Y and Z axis orientation and location of the IMU device is fixed in space. IMU data for all subsequent observations are produced relative to that starting orientation and location. Motion in the X, Y and Z axis of the IMU corresponds to the subject's medio/lateral, anterior/posterior and vertical motion, respectively.

The 3-dimensional motion data from each subject-performed task will be collected for further analysis, including a range of postural stability measures, a sensory adaptability analysis, a sensory integration analysis, an analysis of anterior/posterior, medio/lateral, and vertical motion, and a range of other frequency and amplitude measures.

Included in the preferred embodiment of the analysis methodology is (i) an assessment of the validity of subject's test data (i.e. did the subject attempt to perform the test to the best of their abilities or did they try to manipulate their motion), and (ii) an assessment of the potential stability risk of the subject under yet more challenging motor tasks.

These analyses quantify the subject's postural stability, quantify the adaptability of the subject's visual, somatosensory and vestibular systems, and identify potential sensory integration shortfalls—information which may inform patient diagnosis and physician treatment decisions.

The method for analysis of postural stability involves the calculation of a multitude of indicative statistics, including the following:

For each time sample collected, we calculate:

A_{VM}=√((A_{X})²+(A_{Y})²+(A_{Z})²); and A_{VMF}=√((A_{XF})²+(A_{YF})²+(A_{ZF})²)

Where:

A_{VM}=Acceleration Vector Magnitude;

A_{VMF}=Acceleration Vector Magnitude, post-filter;

A_{X}=The component of linear acceleration as measured along the X axis;

A_{XF}=The post-filter component of linear acceleration as measured along the X axis;

A_{Y}=The component of linear acceleration as measured along the Y axis;

A_{YF}=The post-filter component of linear acceleration as measured along the Y axis;

A_{Z}=The component of linear acceleration as measured along the Z axis; and

A_{ZF}=The post-filter component of linear acceleration as measured along the Z axis.

For each time series associated with a specific motor task, we calculate summary statistics:

For the entire time series less the first “k”-seconds of data, summary statistics are calculated, including the maximum (“MAX”), minimum (“MIN”), mean (“MEAN”), median (“MED”), standard deviation (“SD”) and variance (“VAR”) of A_{VM}, A_{VMF}, A_{X}, A_{XF}, A_{Y}, A_{YF}, A_{Z} and A_{ZF}.

In the preferred embodiment, k=3 seconds; in other embodiments, k can range from zero seconds to 30 seconds.

For the entire time series less the first k-seconds of data, a fast Fourier transform (“FFT”) algorithm is performed on each time series of A_{VM}, A_{X}, A_{Y} and A_{Z}; following the FFT calculations, a spectral centroid is determined for each of A_{VM}, A_{X}, A_{Y} and A_{Z} as SC_{VM}, SC_{X}, SC_{Y} and SC_{Z}, respectively. In the preferred embodiment, k=3 seconds; in other embodiments, k can range from zero seconds to 30 seconds.

For each time series associated with a specific motor task, we calculate volumetric statistics:

For the entire time series less the first k-seconds of data, the volume of an ellipsoid where the radii are the SD of each of A_{XF}, A_{YF}, and A_{ZF}:

V_{T}=4/3π*SD A_{XF}*SD A_{YF}*SD A_{ZF}.

Where V_{T}=Volume of the ellipsoid for the time series (less the first k-seconds of data).

For each time series associated with a specific motor task, we calculate time-window analysis statistics:

For the entire time series, we calculate the A_{VMF} MEAN, MED, SD, and VAR associated with several time-window analyses of the data; each time-window is identified by the amount of time (“p”) associated with the analysis (i.e. for a “4-second window analysis”, p=4).

For each time-window analysis, we calculate the A_{VMF} MAX, MIN, MEAN, MED, SD and VAR for each subset in a time progression of subsets subsumed within the entire time series of data (with each subset having a time-duration of “p” seconds).

For the first data subset, the time-window analysis is conducted on the data starting with the first data observation after k-seconds of data (at data point k+1) and ends p-seconds thereafter (at data point “m”); for the second data subset, the time-window analysis is conducted on the data starting at data point k+2 and ends at data point m+1; for the n^{th} data subset, the time-window analysis is conducted on the data starting at data point k+n and ends at data point m+(n−1). The last data subset included in the analysis is the subset for which m+(n−1) is the last data point in the time series.

An A_{VMF} MEAN, MED, SD and VAR is calculated for the subsets' A_{VMF} MAX, MIN, MEAN, SD and VAR.

Using the same time-window analysis methodology described above, each of the V_{T} MEAN, MED, SD and VAR is calculated for several time-window analyses of the data.

For each motor task associated with a specific subject (person), we calculate a “postural stability” score relative to a selected cohort or peer group:

From the Global Database of collected information, a specific peer group may be formed by sorting the database by one or more characteristics collected for each subject (such as age, gender, height, weight, health factor, etc.); for the selected peer group, the MEAN and SD values are calculated for each of the SD of A_{VMF} (the “Amplitude Measure”) and the SC_{VM} (the “Frequency Measure”) for each test (such as TLEO, TLEC, TSEO, TSEC, TLEOFP, TLECFP, TSEOFP, TSECFP, and potentially others).

For each such measure, the peer group MEAN, +/−1SD and +/−2SD will each be assigned an ordinal value. In the preferred embodiment, the peer groups will be selected from healthy subjects and the MEAN will be assigned an ordinal value of 85; +1 SD and −1 SD will be assigned values of 90 and 80, respectively; +2 SD and −2 SD will be assigned values of 95 and 75, respectively; no score can exceed 100 nor be less than zero.

Based on the selected peer group curve, an ordinal value is assigned to each of the Amplitude Measure and the Frequency Measure for each motor task associated with a specific subject. The average of the ordinal values for the Amplitude Measure and the Frequency Measure associated with a specific motor task is calculated as the “Combined Measure”. Each such ordinal value will also be assigned an interval value. In the preferred embodiment, ordinal values of zero through 59 will have an interval value of “F”; ordinal values of 60 through 69 will have an interval value of “D”; ordinal values of 70 through 72 will have and interval value of “C−”; ordinal values of 73 through 76 will have an interval value of “C”; ordinal values of 77 through 79 will have and interval value of “C+”; ordinal values of 80 through 82 will have and interval value of “B−”; ordinal values of 83 through 86 will have an interval value of “B”; ordinal values of 87 through 89 will have and interval value of “B+”; ordinal values of 90 through 92 will have and interval value of “A−”; ordinal values of 93 through 96 will have an interval value of “A”; ordinal values of 97 through 100 will have and interval value of “A+”.

For each motor task associated with a specific subject (person), we screen the postural stability scores for possible test manipulation by the subject:

Based on the selected peer group curve, the ordinal values assigned to each of the Amplitude Measure, the Frequency Measure and the Combined Measure are evaluated for possible test manipulation by the subject; motor task scores below a threshold level will require that the subject (if otherwise healthy) retake the test. In the preferred embodiment, motor task scores for the Amplitude Measure and the Frequency Measure which are assigned an ordinal value of less than 70 for healthy subjects will be indicative of possible test manipulation.

For each motor task associated with a specific subject (person), we screen the postural stability scores for possible stability risks:

Based on the selected peer group curve, the ordinal values assigned to each of the Amplitude Measure, the Frequency Measure and the Combined Measure are evaluated for possible stability risks associated with more difficult motor tests; test scores below a threshold level will require the approval by the test administrator before the subject attempts the next, more difficult motor task. In the preferred embodiment, test scores for the Amplitude Measure and the Frequency Measure which are assigned an ordinal value of less than 70 will be indicative of possible stability risks.

For each subject, we calculate a “basic stability” score relative to a selected cohort or peer group:

Using the per-test ordinal values assigned above for tests TLEO, TLEC, TSEO and TLEOFP, a weighted average “basic stability” score is calculated for each of the Amplitude Measures, the Frequency Measures and the Combined Measures; for each, an ordinal and interval value is assigned as per the methodology described above. In the preferred embodiment, the weighting of each test is equal.

For each subject, we calculate a “challenged stability” score relative to a selected cohort or peer group:

Using the per-test ordinal values assigned above for tests TSEC, TLECFP, TSEOFP and TSECFP, a weighted average “challenged stability” score is calculated for each of the Amplitude Measures, the Frequency Measures and the Combined Measures; for each, an ordinal and interval value is assigned as per the methodology described above. In the preferred embodiment, the weighting of each test is equal.

For each subject, we calculate a “basic-to-challenged adaptability” score:

Using the “basic stability” and “challenged stability” ordinal scores calculated above, a “basic-to-challenged adaptability” score is calculated as the difference of “challenged stability” less “basic stability”.

For this measure, the peer group MEAN, +/−1SD and +/−2SD for each of the Amplitude Measure, the Frequency Measure and the Combined Measure will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero.

Based on the selected peer group curve, an ordinal value is assigned to each of the subject's “basic-to-challenged adaptability” scores. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For each subject, we calculate a “composite stability” score relative to a selected cohort or peer group:

Using the per-test ordinal and interval values assigned above, a weighted average composite balance score is calculated for each of the Amplitude Measures, the Frequency Measures and the Combined Measures. In the preferred embodiment, the weighting of each test is equal.

In cases where a person is periodically retested, a pre-injury Mi Balance composite stability “baseline” is calculated as the subject's best composite stability test score.

For each subject, we calculate a “visual adaptability to change” statistic:

With regard to the selected peer group: for each of the Amplitude Measures, the Frequency Measures and the Combined Measures, the MEAN and SD is calculated for the weighted average difference of ordinal values for (TSEO-TLEO), (TSEOFP-TLEOFP), (TLEOFP-TLEO), and (TSEOFP-TSEO).

The MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero; further, the weighting is equal.

For the subject, the weighted average difference of ordinal values for each of the Amplitude Measures, the Frequency Measures and the Combined Measures for (TSEO-TLEO), (TSEOFP-TLEOFP), (TLEOFP-TLEO), and (TSEOFP-TSEO) is calculated.

Based on the selected peer group curve, an ordinal value is assigned to each of the subject's “visual adaptability to change” scores. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For each subject, we calculate a “vestibular adaptability to change” statistic:

With regard to the selected peer group: for each of the Amplitude Measures, the Frequency Measures and the Combined Measures, the MEAN and SD is calculated for the weighted average difference of ordinal values for (TLEC-TLEO), (TLECFP-TLEOFP), (TLEOFP-TLEO), and (TLECFP-TLEC).

The MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero; further, the weighting is equal.

For the subject, and for each of the Amplitude Measures, the Frequency Measures and the Combined Measures, the weighted average difference of ordinal values for (TLEC-TLEO), (TLECFP-TLEOFP), (TLEOFP-TLEO), and (TLECFP-TLEC) is calculated.

Based on the selected peer group curve, an ordinal value is assigned to each of the subject's “visual adaptability to change” scores. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For each subject, we calculate a “somatosensory adaptability to change” statistic:

With regard to the selected peer group: for each of the Amplitude Measures, the Frequency Measures and the Combined Measures, the MEAN and SD is calculated for the weighted average difference of ordinal values for (TLEC-TLEO), (TSEC-TSEO), (TSEO-TLEO), and (TSEC-TLEC).

The MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero; further, the weighting is equal.

For the subject, for each of the Amplitude Measures, the Frequency Measures and the Combined Measures, the weighted average difference of ordinal values for (TLEC-TLEO), (TSEC-TSEO), (TSEO-TLEO), and (TSEC-TLEC) is calculated.

Based on the selected peer group curve, an ordinal value is assigned to each of the subject's “visual adaptability to change” scores. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For each subject, we calculate a “vision and vestibular integrated adaptability to change” statistic:

With regard to the selected peer group: for the Amplitude Measures, the Frequency Measures and the Combined Measures, the MEAN and SD is calculated for the weighted average difference of ordinal values for (TLEOFP-TLEO), (TLECFP-TLEC), (TSEOFP-TSEO), and (TSECFP-TSEC).

The MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero; further, the weighting is equal.

For the subject, the weighted average difference of ordinal values for each of the Amplitude Measures, the Frequency Measures and the Combined Measures for (TLEOFP-TLEO), (TLECFP-TLEC), (TSEOFP-TSEO), and (TSECFP-TSEC) is calculated.

Based on the selected peer group curve, an ordinal value is assigned to each of the subject's “vision and vestibular integrated adaptability to change” scores. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For each subject, we calculate a “vision and somatosensory integrated adaptability to change” statistic:

With regard to the selected peer group: for the Amplitude Measures, the Frequency Measures and the Combined Measures, the MEAN and SD is calculated for the weighted average difference of ordinal values for (TSEO-TLEO), (TSEC-TLEC), (TSEOFP-TLEOFP), and (TSECFP-TLEOFP). The MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero; further, the weighting is equal.

For the subject, the weighted average difference of ordinal values for (TSEO-TLEO), (TSEC-TLEC), (TSEOFP-TLEOFP), and (TSECFP-TLEOFP) is calculated.

Based on the selected peer group curve, an ordinal value is assigned to each of the subject's “vision and somatosensory integrated adaptability to change” scores. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For each subject, we calculate a “vestibular and somatosensory integrated adaptability to change” statistic:

With regard to the selected peer group: for the Amplitude Measures, the Frequency Measures and the Combined Measures, the MEAN and SD is calculated for the weighted average difference of ordinal values for (TLEC-TLEO), (TSEC-TSEO), (TLECFP-TLEOFP), and (TSECFP-TSEOFP).

The MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero; further, the weighting is equal.

For the subject, the weighted average difference of ordinal values for (TLEC-TLEO), (TSEC-TSEO), (TLECFP-TLEOFP), and (TSECFP-TSEOFP) is calculated.

Based on the selected peer group curve, an ordinal value is assigned to each of the subject's “vestibular and somatosensory integrated adaptability to change” scores. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For each time series associated with a specific motor task, we calculate stability strategy statistics:

For the entire time series less the first k-seconds of data, the anterior/posterior component of motion is calculated as a percentage of total motion:

Test Specific A/P Amplitude Percentage=SD A_{XF}/SD A_{VMF}; and

Test Specific A/P Frequency=SC A_{X}.

For the entire time series less the first k-seconds of data, the medio/lateral component of motion is calculated as a percentage:

Test Specific M/L Amplitude Percentage=SD A_{ZF}/SD A_{VMF}; and

Test Specific M/L Frequency=SC A_{Z}.

For the entire time series less the first k-seconds of data, the vertical component of motion is calculated as a percentage:

Test Specific VERT Amplitude Percentage=SD A_{YF}/SD A_{VMF}; and

Test Specific VERT Frequency=SC A_{Y}.

For the time series' associated with all motor tasks, we calculate the subject's aggregate stability strategy statistics:

The “Anterior/Posterior Motion Percentage” is calculated as the weighted average of the Test Specific A/P Amplitude Percentages from each of TLEO, TLEC, TSEO, TSEC, TLEOFP, TLECFP, TSEOFP, and TSECFP; similarly, the “Anterior/Posterior Mean Frequency” is calculated as the weighted average of the Test Specific A/P Frequencies from each of TLEO, TLEC, TSEO, TSEC, TLEOFP, TLECFP, TSEOFP, and TSECFP. In the preferred embodiment, the weighting for each measure is equal.

For these measures, the peer group MEAN, +/−1SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero.

Based on the selected peer group curve, an ordinal value is assigned to each of the subject's Anterior/Posterior Motion Percentage score and Anterior/Posterior Mean Frequency score. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

The “Medio/Lateral Motion Percentage” is calculated as the weighted average of the Test Specific M/L Amplitude Percentages from each of TLEO, TLEC, TSEO, TSEC, TLEOFP, TLECFP, TSEOFP, and TSECFP; similarly, the “Medio/Lateral Mean Frequency” is calculated as the weighted average of the Test Specific M/L Frequencies from each of TLEO, TLEC, TSEO, TSEC, TLEOFP, TLECFP, TSEOFP, and TSECFP. In the preferred embodiment, the weighting for each measure is equal.

For these measures, the peer group MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero.

Based on the selected peer group curve, an ordinal value is assigned to each of the subject's Medio/Lateral Motion Percentage score and Medio/Lateral Mean Frequency score. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

The “Vertical Motion Percentage” is calculated as the weighted average of the Test Specific M/L Amplitude Percentages from each of TLEO, TLEC, TSEO, TSEC, TLEOFP, TLECFP, TSEOFP, and TSECFP; similarly, the “Vertical Mean Frequency” is calculated as the weighted average of the Test Specific VERT Frequencies from each of TLEO, TLEC, TSEO, TSEC, TLEOFP, TLECFP, TSEOFP, and TSECFP. In the preferred embodiment, the weighting for each measure is equal.

For these measures, the peer group MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero.

Based on the selected peer group curve, an ordinal value is assigned to each of the subject's Vertical Motion Percentage score and Vertical Mean Frequency score. These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

### Generation of Mi Balance Report:

Following the calculations described above, a “Mi Balance” postural stability analysis report (1100) is generated relative to the subject.

In the preferred embodiment, the Mi Balance postural stability analysis report contains the ordinal and/or interval scores for each testing date for each of the following Combined Measures: TLEO, TLEC, TSEO, TSEC, TLEOFP, TLECFP, TSEOFP, TSECFP, Basic Stability, Challenged Stability, Basic-to-Challenged Stability, Composite Stability, Visual Adaptability to Change, Vestibular Adaptability to Change, Somatosensory Adaptability to Change, Vision and Vestibular Adaptability to Change, Vision and Somatosensory Adaptability to Change, and Vestibular and Somatosensory Adaptability to Change; and each of the following Amplitude Measures: Anterior/Posterior Motion Percentage, Medio/Lateral Motion Percentage, and Vertical Motion Percentage.

In other embodiments, these and/or other measures or scores referenced above are contained in the Mi Balance postural stability analysis report.

### Mi Integrated Performance

The Mi Integrated Performance component of the invention is a system and method for administering and scoring certain dual-task tests used to evaluate a person's cognitive abilities while their postural stability is challenged. The cognitive testing and postural stability testing components associated with Mi Integrated Performance occur contemporaneously. Each of these components are described more fully below:

### Cognitive Testing Component

As with Mi Thinking, the cognitive testing component of the Mi Integrated Performance system evaluates elements of a person's cognitive abilities and changes in those cognitive abilities over time.

The system administers and scores one or more neuropsychological tests; all such tests are derivations of one or more similar tests for which, in clinical evaluations, human subjects have exhibited lowered neuropsychological performance following concussion injuries. Examples of such tests include: the Trail-Making Test, Parts A & B; the Digit Span Test, Forward and Backward (from the Wechsler Adult Intelligence Scale); and the Stroop Task. Further, the cognitive testing component of Mi Integrated Performance involves one or more tests or subsets of tests utilized in the Mi Thinking component of the invention.

In the preferred embodiment, the administration and scoring of the cognitive tests will be conducted electronically through subject interaction with software resident on a computer while the subject is engaged in a physically challenging task such as TSEO (1700); software resident on the computer (110) will calculate the person's cognitive test score(s); cognitive test data will be transmitted to the Global Database (114); certain elements of the Global Database will be accessible by the computer for comparative analysis.

The objective methods used to score the test(s) will be dependent on the nature of the test(s), but will generally include one or more timed tasks and a may include other criteria. In cases where a person is periodically retested, a pre-injury “baseline” for the cognitive testing component of Mi Integrated Performance is calculated as the subject's best cognitive test score (i.e. in the case of a test scoring rubric which measures elapsed time, the shortest time to complete the test will be the subject's pre-injury baseline score).

For each cognitive testing component of Mi Integrated Performance associated with a specific subject (person), we calculate a score relative to a selected cohort or peer group:

From the Global Database of collected information, a specific peer group may be formed by sorting the database by one or more characteristics collected for each subject (such as age, gender, height, weight, health factor, etc.); for the selected peer group, the mean (“MEAN”) and standard deviation (“SD”) values are calculated for each of the test scoring criteria (such as elapsed time) for each test.

For each test scoring criteria, the peer group MEAN, +/−1 SD and +/−2SD will each be assigned an ordinal value. In the preferred embodiment, the peer groups will be selected from healthy subjects and the MEAN will be assigned an ordinal value of 85; +1 SD and −1 SD will be assigned values of 90 and 80, respectively; +2 SD and −2 SD will be assigned values of 95 and 75, respectively; no score can exceed 100 nor be less than zero.

Based on the selected peer group curve, an ordinal value is assigned to each of the scoring criteria values for each cognitive test associated with a specific subject. Each such ordinal value will also be assigned an interval value. In the preferred embodiment, ordinal values of zero through 59 will have an interval value of “F”; ordinal values of 60 through 69 will have an interval value of “D”; ordinal values of 70 through 72 will have and interval value of “C−”; ordinal values of 73 through 76 will have an interval value of “C”; ordinal values of 77 through 79 will have and interval value of “C+”; ordinal values of 80 through 82 will have and interval value of “B−”; ordinal values of 83 through 86 will have an interval value of “B”; ordinal values of 87 through 89 will have and interval value of “B+”; ordinal values of 90 through 92 will have and interval value of “A−”; ordinal values of 93 through 96 will have an interval value of “A”; ordinal values of 97 through 100 will have and interval value of “A+”.

### Generation of Mi Integrated Performance—Cognitive Abilities Analysis Report

Following the calculations described above, a “Mi Integrated Performance—Cognitive Abilities Analysis” report is generated relative to the subject.

In the preferred embodiment, the “Mi Integrated Performance—Cognitive Abilities Analysis” report contains the ordinal and/or interval scores for each testing date and for each of the administered cognitive tests; further, this report will display a comparative analysis of the cognitive tests or subsets of tests executed in Mi Integrated Performance (in a dual-task condition) versus those same tests or subsets of tests executed during Mi Thinking (in a single-task condition) and as performed during the same Mi CARE System testing session.

For the current testing session, a Mi Integrated Performance—Cognitive Abilities Composite Score is calculated as the weighted average of the ordinal scores associated with each Mi Integrated Performance cognitive test; in the preferred embodiment, the weighting is equal.

### Postural Stability Testing Component

As with Mi Balance testing, the postural stability testing component of the Mi Integrated Performance system measures and records a plurality of inertial motion data while the subject (a person) executes one or more physical tasks. However, for the postural stability testing component of Mi Integrated Performance, the subject will also be executing a cognitive test contemporaneously with their execution of a physical task.

The collected inertial motion data are processed by a connected mobile computer for meaningful analysis and use by trained personnel. The system utilizes one or more inexpensive, non-invasive, portable and wearable inertial motion sensing and reporting units (each an “IMU”) encapsulated within a purpose-built protective enclosure (106 for the wired IMU; 302 for the wireless IMU), an integrated fitment device worn by the subject (104), a computer (110) connected either wirelessly (304) or via cable interface (108) to the IMU(s), software used to calculate parameters associated with a person's postural stability, a central database of collected data and processed information (the Global Database) (114)) accessible by the computer (110), and, for certain tests, a foam pad (202).

In one embodiment, the IMU includes a tri-axial accelerometer (502), tri-axial gyroscope (504), tri-axial magnetometer (506), an embedded microprocessor (508) and a USB port (510) (collectively, the “Wired-IMU” (500)). The Wired-IMU is connected to a mobile computer via cable interface (108).

In another embodiment, the IMU also includes a wireless communications module (606), a battery (604) and a battery charger (602) (collectively, the “Wireless-IMU” (600)).

The Wireless-IMU is connected to a mobile computer through wireless communications such as Bluetooth or other similar technology.

The IMU is housed in a purpose-build protective enclosure (1200) and attached to a purpose-built fitment device (104); in the preferred embodiment, the purpose-built fitment device is a belt that can be adjusted to fit a most subject waist sizes. In the preferred embodiment of the methodology, the IMU, which is housed in a protective enclosure, is to be securely attached to the subject using the fitment device, near the subject's center of mass (in the center of the lower back, approximately at the 5^{th} lumbar vertebrae).

The IMU samples certain data, preferably at over 1,000 Hz (702), before application of a Kalman filter (704); sensor data is available in excess of 240 Hz post-filter and includes: a timestamp, Quaternion X (“Q_{X}”), Quaternion Y (“Q_{Y}”), Quaternion Z (“Q_{Z}”), Quaternion W (“Q_{W}”), Acceleration X (“A_{X}”), Acceleration Y (“A_{Y}”), Acceleration Z (“A_{Z}”), Gyroscope X (“G_{X}”), Gyroscope Y (“G_{Y}”), Gyroscope Z (“G_{Z}”), Compass X (“C_{X}”), Compass Y (“C_{Y}”), Compass Z (“C_{Z}”) (collectively, the “Processed Data”).

The Processed Data is then transmitted (708) to the computer. For certain calculations, A_{X}, A_{Y} and A_{Z} are subject to additional filtering on the computer, resulting in A_{XF}, A_{YF} and A_{ZF}; in the preferred embodiment, this additional filtering consists of a first-order, low-pass Butterworth filter at 20 Hz.

Certain biometric and identifying data associated with the test subjects will be collected and stored in the Global Database; while wearing an IMU connected to a mobile computer, subjects will be asked to perform one or more tasks which test their postural stability while they are simultaneously engaged in the Cognitive Testing component of Mi Integrated Performance testing; data collected will be stored in the Global Database; a comprehensive report will be provided to the subject and/or the test administrator.

In the preferred embodiment of the testing methodology, IMU data is collected while a subject performs a single motor task for the duration of each Cognitive Test component of the dual-task testing. In the preferred embodiment, the motor task is TSEO (1700). In other embodiments of the testing methodology, one or more of the previously identified eight motor tasks are performed by the subject; in yet other embodiments of the testing methodology, the IMU may collect data while the subject is walking, running or performing some other motor task.

Prior to performing each motor task, a “tare function” is executed whereby the starting X, Y and Z axis orientation and location of the IMU device is fixed in space. IMU data for all subsequent observations are produced relative to that starting orientation and location. Motion in the X, Y and Z axis of the IMU corresponds to the subject's medio/lateral, anterior/posterior and vertical motion, respectively.

The 3-dimensional motion data from each subject-performed task will be collected for further analysis, including a range of postural stability measures, a sensory adaptability analysis, a sensory integration analysis, an analysis of anterior/posterior, medio/lateral, and vertical motion, and a range of other frequency and amplitude measures.

Included in the preferred embodiment of the analysis methodology is (i) an assessment of the validity of subject's test data (i.e. did the subject attempt to perform the test to the best of their abilities or did they try to manipulate their motion), and (ii) an assessment of the potential stability risk of the subject under yet more challenging motor tasks.

These analyses quantify the subject's postural stability while engaged in dual-task testing—information which may inform patient diagnosis and physician treatment decisions.

The method for analysis of postural stability involves the calculation of a multitude of indicative statistics, including the following:

For each time sample collected, we calculate:

A_{VM}=√((A_{X})²+(A_{Y})²+(A_{Z})²); and A_{VMF}=√((A_{XF})²+(A_{YF})²+(A_{ZF})²)

Where:

A_{VM}=Acceleration Vector Magnitude;

A_{VMF}=Acceleration Vector Magnitude, post-filter;

A_{X}=The component of linear acceleration as measured along the X axis;

A_{XF}=The post-filter component of linear acceleration as measured along the X axis;

A_{Y}=The component of linear acceleration as measured along the Y axis;

A_{YF}=The post-filter component of linear acceleration as measured along the Y axis;

A_{Z}=The component of linear acceleration as measured along the Z axis; and

A_{ZF}=The post-filter component of linear acceleration as measured along the Z axis.

For each time series associated with a specific motor task, we calculate summary statistics:

For the entire time series less the first “k”-seconds of data, summary statistics are calculated, including the maximum (“MAX”), minimum (“MIN”), mean (“MEAN”), median (“MED”), standard deviation (“SD”) and variance (“VAR”) of A_{VM}, A_{VMF}, A_{X}, A_{XF}, A_{Y}, A_{YF}, A_{Z} and A_{ZF}. In the preferred embodiment, k=3 seconds; in other embodiments, k can range from zero seconds to 30 seconds.

For the entire time series less the first k-seconds of data, a fast Fourier transform (“FFT”) algorithm is performed on each time series of A_{VM}, A_{X}, A_{Y} and A_{Z}; following the FFT calculations, a spectral centroid is determined for each of A_{VM}, A_{X}, A_{Y} and A_{Z} as SC_{VM}, SC_{X}, SC_{Y} and SC_{Z}, respectively. In the preferred embodiment, k=3 seconds; in other embodiments, k can range from zero seconds to 30 seconds.

For each time series associated with a specific motor task, we calculate volumetric statistics:

For the entire time series less the first k-seconds of data, the volume of an ellipsoid where the radii are the SD of each of A_{XF}, A_{YF}, and A_{ZF}:

V_{T}=4/3π*SD A_{XF}*SD A_{YF}*SD A_{ZF}.

Where:

V_{T}=Volume of the ellipsoid for the time series (less the first k-seconds of data).

For each time series associated with a specific motor task, we calculate time-window analysis statistics:

For the entire time series, we calculate the A_{VMF} MEAN, MED, SD, and VAR associated with several time-window analyses of the data; each time-window is identified by the amount of time (“p”) associated with the analysis (i.e. for a “4-second window analysis”, p=4).

For each time-window analysis, we calculate the A_{VMF} MAX, MIN, MEAN, MED, SD and VAR for each subset in a time progression of subsets subsumed within the entire time series of data (with each subset having a time-duration of “p” seconds).

For the first data subset, the time-window analysis is conducted on the data starting with the first data observation after k-seconds of data (at data point k+1) and ends p-seconds thereafter (at data point “m”); for the second data subset, the time-window analysis is conducted on the data starting at data point k+2 and ends at data point m+1; for the n^{th} data subset, the time-window analysis is conducted on the data starting at data point k+n and ends at data point m+(n−1). The last data subset included in the analysis is the subset for which m+(n−1) is the last data point in the time series. An A_{VMF} MEAN, MED, SD and VAR is calculated for the subsets' A_{VMF} MAX, MIN, MEAN, SD and VAR.

Using the same time-window analysis methodology described above, each of the V_{T} MEAN, MED, SD and VAR is calculated for several time-window analyses of the data.

For each motor task associated with a specific subject (person), we calculate a “postural stability” score relative to a selected cohort or peer group:

From the Global Database of collected information, a specific peer group may be formed by sorting the database by one or more characteristics collected for each subject (such as age, gender, height, weight, health factor, etc.); for the selected peer group, the MEAN and SD values are calculated for each of the SD of A_{VMF} (the “Amplitude Measure”) and the SC_{VM} (the “Frequency Measure”) for each Postural Stability component of the Mi Integrated Performance testing (such as TSEO and potentially others).

For each such measure, the peer group MEAN, +/−1 SD and +/−2SD will each be assigned an ordinal value. In the preferred embodiment, the peer groups will be selected from healthy subjects and the MEAN will be assigned an ordinal value of 85; +1 SD and −1 SD will be assigned values of 90 and 80, respectively; +2 SD and −2 SD will be assigned values of 95 and 75, respectively; no score can exceed 100 nor be less than zero.

Based on the selected peer group curve, an ordinal value is assigned to each of the Amplitude Measure and the Frequency Measure for each motor task associated with a specific subject.

The average of the ordinal values for the Amplitude Measure and the Frequency Measure associated with a specific motor task is calculated as the “Combined Measure”. Each such ordinal value will also be assigned an interval value. In the preferred embodiment, ordinal values of zero through 59 will have an interval value of “F”; ordinal values of 60 through 69 will have an interval value of “D”; ordinal values of 70 through 72 will have and interval value of “C−”; ordinal values of 73 through 76 will have an interval value of “C”; ordinal values of 77 through 79 will have and interval value of “C+”; ordinal values of 80 through 82 will have and interval value of “B−”; ordinal values of 83 through 86 will have an interval value of “B”; ordinal values of 87 through 89 will have and interval value of “B+”; ordinal values of 90 through 92 will have and interval value of “A−”; ordinal values of 93 through 96 will have an interval value of “A”; ordinal values of 97 through 100 will have and interval value of “A+”.

For each motor task associated with a specific subject (person), we screen the postural stability scores for possible test manipulation by the subject:

Based on the selected peer group curve, the ordinal values assigned to each of the Amplitude Measure, the Frequency Measure and the Combined Measure are evaluated for possible test manipulation by the subject; motor task scores below a threshold level will require that the subject (if otherwise healthy) retake the test. In the preferred embodiment, motor task scores for the Amplitude Measure and the Frequency Measure which are assigned an ordinal value of less than 70 for healthy subjects will be indicative of possible test manipulation.

For each motor task associated with a specific subject (person), we screen the postural stability scores for possible stability risks:

Based on the selected peer group curve, the ordinal values assigned to each of the Amplitude Measure, the Frequency Measure and the Combined Measure are evaluated for possible stability risks associated with more difficult motor tests; test scores below a threshold level will require the approval by the test administrator before the subject attempts the next, more difficult motor task. In the preferred embodiment, test scores for the Amplitude Measure and the Frequency Measure which are assigned an ordinal value of less than 70 will be indicative of possible stability risks.

For each subject, we calculate a “single- to dual-task change” score:

Using the “basic stability” ordinal scores for each of the single-task and dual-task scores calculated above, a “single- to dual-task change” score is calculated as the difference of “basic stability” for the single-task condition less “basic stability” for the dual-task condition.

For this measure, the peer group MEAN, +/−1SD and +/−2SD for each of the Amplitude Measure, the Frequency Measure and the Combined Measure will each be assigned an ordinal value. In the preferred embodiment, the MEAN will be assigned an ordinal value of 0 (zero); +1 SD and −1 SD will be assigned values of 25 and −25, respectively; +2 SD and −2 SD will be assigned values of 50 and −50, respectively; no score can exceed 100 nor be less than −100.

Based on the selected peer group curve, an ordinal value may be assigned to each of the subject's “single- to dual-task change” scores. These ordinal values will also be assigned interval values. In the preferred embodiment, ordinal values of −100 through −50 will have an interval value of “Large Negative Change”; ordinal values of −49 through −25 will have and interval value of “Moderate Negative Change”; ordinal values of −13 through −25 will have an interval value of “Small Negative Change”; ordinal values of −12 through 12 will have and interval value of “Minimal Change”; ordinal values of 13 through 25 will have and interval value of “Small Positive Change”; ordinal values of 26 through 50 will have an interval value of “Moderate Positive Change”; ordinal values of 51 through 100 will have and interval value of “Large Positive Change”.

For each time series associated with a motor task, we calculate stability strategy statistics:

For the entire time series less the first k-seconds of data, the anterior/posterior component of motion is calculated as a percentage of total motion:

Test Specific A/P Amplitude Percentage=SD A_{XF}/SD A_{VMF}; and

Test Specific A/P Frequency=SC A_{X}.

For the entire time series less the first k-seconds of data, the medio/lateral component of motion is calculated as a percentage:

Test Specific M/L Amplitude Percentage=SD A_{ZF}/SD A_{VMF}; and

Test Specific M/L Frequency=SC A_{Z}.

For the entire time series less the first k-seconds of data, the vertical component of motion is calculated as a percentage:

Test Specific VERT Amplitude Percentage=SD A_{YF}/SD A_{VMF}; and

Test Specific VERT Frequency=SC A_{Y}.

For the time series' associated with a motor task, we calculate the subject's aggregate stability strategy statistics:

The “Anterior/Posterior Motion Percentage” is calculated as the weighted average of the Test Specific A/P Amplitude Percentages from each Postural Stability component of Mi Integrated Performance testing; similarly, the “Anterior/Posterior Mean Frequency” is calculated as the weighted average of the Test Specific A/P Frequencies from each Postural Stability component of Mi Integrated Performance testing. In the preferred embodiment, the weighting for each measure is equal.

For these measures, the peer group MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero.

Based on the selected peer group curve, an ordinal value is assigned to each of the subject's Anterior/Posterior Motion Percentage score and Anterior/Posterior Mean Frequency score.

These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

The “Medio/Lateral Motion Percentage” is calculated as the weighted average of the Test Specific M/L Amplitude Percentages from each Postural Stability component of Mi Integrated Performance testing; similarly, the “Medio/Lateral Mean Frequency” is calculated as the weighted average of the Test Specific M/L Frequencies from each Postural Stability component of Mi Integrated Performance testing. In the preferred embodiment, the weighting for each measure is equal.

For these measures, the peer group MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero.

Based on the selected peer group curve, an ordinal value is assigned to each of the subject's Medio/Lateral Motion Percentage score and Medio/Lateral Mean Frequency score.

These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

The “Vertical Motion Percentage” is calculated as the weighted average of the Test Specific M/L Amplitude Percentages from each Postural Stability component of Mi Integrated Performance testing; similarly, the “Vertical Mean Frequency” is calculated as the weighted average of the Test Specific VERT Frequencies from each Postural Stability component of Mi Integrated Performance testing. In the preferred embodiment, the weighting for each measure is equal.

For these measures, the peer group MEAN, +/−1 SD and +/−2SD will each be assigned an ordinal value. In the preferred embodiment, the MEAN will be assigned an ordinal value of 50; +1 SD and −1 SD will be assigned values of 40 and 60, respectively; +2 SD and −2 SD will be assigned values of 30 and 70, respectively; no score can exceed 100 nor be less than zero.

Based on the selected peer group curve, an ordinal value is assigned to each of the subject's Vertical Motion Percentage score and Vertical Mean Frequency score.

These ordinal values will also be assigned interval values. In the preferred embodiment, ordinal values of zero through 19 will have an interval value of “Very Low”; ordinal values of 20 through 29 will have an interval value of “Low”; ordinal values of 30 through 39 will have and interval value of “Below Average”; ordinal values of 40 through 44 will have an interval value of “Average −”; ordinal values of 45 through 54 will have and interval value of “Average”; ordinal values of 55 through 59 will have and interval value of “Average +”; ordinal values of 60 through 69 will have an interval value of “Above Average”; ordinal values of 70 through 79 will have and interval value of “High”; and, ordinal values of 80 through 100 will have an interval value of “Very High”.

For the current testing session, a “Mi Integrated Performance—Postural Stability Composite Score” is calculated as the weighted average of the postural stability ordinal scores associated with each Mi Integrated Performance postural stability test; in the preferred embodiment, the weighting is equal.

### Generate Mi Integrated Performance—Postural Stability Analysis Report

Following the calculations described above, a “Mi Integrated Performance—Postural Stability Analysis” report is generated relative to the subject. In the preferred embodiment, the Mi Integrated Performance—Postural Stability Analysis report contains the Mi Integrated Performance—Postural Stability Composite Score and a comparative analysis including the ordinal and/or interval scores for each testing date for each of the following Combined Measures: TSEO (single-task), and TSEO (dual-task); and each of the following Amplitude Measures: Anterior/Posterior Motion Percentage, Medio/Lateral Motion Percentage, and Vertical Motion Percentage. In other embodiments, these and/or other measures or scores referenced above are contained in the Mi Integrated Performance—Postural Stability Analysis report.

### Combined Dual-Task Calculations and Reporting

Following the generation of the Mi Integrated Performance—Cognitive Abilities Analysis and the Mi Integrated Performance—Postural Stability Analysis, a combined “Mi Integrated Performance Score” is calculated as the weighted average of the Mi Integrated Performance—Postural Stability Composite Score and the Mi Integrated Performance—Cognitive Abilities Composite Score; in the preferred embodiment, the weighting is equal. An aggregate “Mi Integrated Performance” report is generated relative to the subject containing the Mi Integrated Performance Score for the current testing date and each previous testing date (2200).

### Mi Evaluation

The Mi Evaluation component of the invention summarizes current and prior data from Mi Symptoms, Mi Thinking, Mi Balance and Mi Integrated Performance to facilitate the clinical diagnosis of concussion injuries, inform treatment and response strategies, and guide return to play (or return to duty) decisions.

For the current testing session and for each prior testing session, the summary data includes the Mi Symptoms Summative Score, the Mi Thinking Composite Score, the Mi Balance Composite Stability Score, and the Mi Integrated Performance Score.

In the preferred embodiment of the invention, the summary data is displayed on a four-sided, diamond-shaped graph (1900) where, for three of the measures (Mi Balance, Mi Thinking and Mi Integrated Performance), the center of the diagram represents a score of zero and the respective points of the diamond represent scores of 100; for the data axis representing Mi Symptoms, the point of the diamond will represent a score of zero and the center of the graph will represent a score of 72; this data may also be represented in tabular form. The detailed reports from each of Mi Symptoms, Mi Thinking, Mi Balance and Mi Integrated Performance are displayed or printed with the Mi Evaluation summary report.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of assessing a subject for concussion comprising the steps of:
a) collecting self-reported symptoms to produce an Mi symptoms summative score;
b) administering and scoring of at least one cognitive test to produce an Mi thinking composite score;
c) administering and scoring of at least one postural stability test to produce an Mi balance composite stability score;
d) administering and scoring of at least one dual-task test to produce an Mi integrated performance score;
e) summarizing the Mi symptoms summative score, Mi thinking composite score, Mi balance composite stability score and Mi integrated performance score, to create an Mi evaluation summary report; and
f) displaying the Mi evaluation summary report.

2. The method of claim 1, in which the step (a) of producing the Mi symptoms summative score comprises the steps of:
i) collecting and storing symptom data based on a plurality of symptoms reported on a checklist received from the subject;
ii) grading the checklist by assigning a grade to each of the plurality of symptoms reported on the checklist; and
iii) calculating the Mi symptoms summative score from the grades assigned to each of the plurality of symptoms.

3. The method of claim 2, further comprising the step of generating a symptoms analysis report comprising the plurality of symptoms reported by the subject, the grades assigned to each of the symptoms, and the Mi symptoms summative score.

4. The method of claim 1, in which the step (b) of producing the Mi thinking composite score comprises the steps of:
i) determining a peer group for the subject based on characteristics of the subject;
ii) administering a cognitive test having test scoring criteria to the subject;
iii) assigning subject test scores to the subject for the test scoring criteria of the cognitive test;
iv) retrieving peer group test scores from a database for the test scoring criteria for the cognitive test as administered to the peer group;
v) calculating a peer group curve comprising mean and standard deviation values for the peer group test scores;
vi) assigning an ordinal value to the mean and standard deviation values for the peer group test scores;
vii) based on the peer group curve, assigning an ordinal value to the subject test scores;
viii) repeating steps (ii) through (vii) until all cognitive tests have been administered to the subject, then
ix) calculating the Mi thinking composite score from a weighted average of the ordinal values from step (vii) for all of the cognitive tests.

5. The method of claim 4, in which the ordinal values are between 0 and 100.

6. The method of claim 4, further comprising the step of assigning an interval value to the Mi thinking composite score.

7. The method of claim 6, in which the interval value is between A+ and F.

8. The method of claim 4, in which the cognitive tests are selected from the group consisting of derivations of the trail-making test, parts A & B; the Wechsler adult intelligence scale digit span test, forward and backward; and the Stroop task.

9. The method of claim 1, in which the step (c) of producing the Mi balance composite stability score comprises the steps of:
i) determining a peer group for the subject based on characteristics of the subject;
ii) administering a postural stability test to the subject comprising a plurality of motor tasks, each motor task having a specified duration;
iii) collecting data from an inertial motion sensing and reporting unit worn by the subject during the postural stability test;
iv) processing the data from the inertial motion sensing and reporting unit to produce subject processed data, and storing the subject processed data in a database;
v) calculating indicative postural stability statistics from the subject processed data;
vi) retrieving peer group postural stability statistics for the postural stability test as administered to the peer group from a database;
vii) calculating a peer group amplitude measure and frequency measure, comprising mean and standard deviation values for the peer group postural stability statistics;
viii) assigning an ordinal value to the mean and standard deviation values for the peer group postural stability statistics;
ix) based on mean and standard deviation values for the peer group postural stability statistics relative to the indicative postural stability statistics, calculating a postural stability score for the subject;
x) assigning an ordinal value to the postural stability score for the subject;
xi) for at least some of the plurality of motor tasks, calculating a basic stability score for the ordinal value assigned to the subject relative to the peer group for each of amplitude measures, frequency measures and combined measures for each task;
xii) for at least some of the plurality of motor tasks, calculating a challenged stability score for the ordinal value assigned to the subject relative to the peer group for each of amplitude measures, frequency measures and combined measures for each task;
xiii) calculating a basic-to-challenged adaptability score, calculated as the difference of the basic stability score and the challenged stability score for selected motor tasks;
xiv) calculating a weighted average composite stability score for the subject relative to the peer group;
xv) calculating a visual adaptability to change statistic for the subject relative to the peer group weighted average difference of ordinal values for selected motor tasks;
xvi) calculating a vestibular adaptability to change statistic for the subject relative to the peer group weighted average difference of ordinal values for selected motor tasks;
xvii) calculating a somatosensory adaptability to change statistic for the subject relative to the peer group weighted average difference of ordinal values for selected motor tasks;
xviii) calculating a vision and vestibular integrated adaptability to change statistic for the subject relative to the peer group weighted average difference of ordinal values for selected motor tasks;
xix) calculating a vision and somatosensory integrated adaptability to change statistic for the subject relative to the peer group weighted average difference of ordinal values for selected motor tasks;
xx) calculating a vestibular and somatosensory integrated adaptability to change statistic for the subject relative to the peer group weighted average difference of ordinal values for selected motor tasks;
xxi) for each time series associated with a motor task, calculating stability strategy statistics for the subject;
xxii) for the time series associated with all motor tasks, calculating an aggregate stability strategy statistics for the subject; and
xxiii) calculating the Mi balance composite stability score from a weighted average of values from steps (ii) through (xxii).

10. The method of claim 9, further comprising the step of assessing validity of the subject's test data by screening the postural stability statistics for possible test manipulation by the subject.

11. The method of claim 9, further comprising the step of assessing potential stability risk of the subject under more challenging motor tasks by screening the postural stability statistics for possible stability risks.

12. The method of claim 11, in which the specified duration for each motor task is 30 seconds.

13. The method of claim 11, in which there are eight motor tasks.

14. The method of claim 11, in which the motor tasks are selected from the group consisting of two legs, eyes open, firm surface; two legs, eyes closed, firm surface; tandem stance, eyes open, firm surface; tandem stance, eyes closed, firm surface; two legs, eyes open, foam pad; two legs, eyes closed, foam pad; tandem stance, eyes open, foam pad; and tandem stance, eyes closed, foam pad.

15. The method of claim 11, in which the step (v) of calculating indicative postural stability statistics comprises:
A) for each time sample within a time series associated with each motor task, calculate an acceleration vector magnitude from components of linear acceleration along X, Y and Z axes;
B) for each time series associated with each motor task:
1) calculate summary statistics from the acceleration vector magnitudes for each of the time samples in the motor task;
2) perform a fast Fourier transform on each time series of the acceleration vector magnitude and the components of linear acceleration along X, Y and Z axes, to determine a spectral centroid for each of the acceleration vector magnitude and the components of linear acceleration along X, Y and Z axes;
3) calculate volumetric statistics comprising the volume of an ellipsoid where the radii are the standard deviations of each of the components of linear acceleration along X, Y and Z axes;
4) for the entire time series, calculate time-window analysis statistics for a plurality of time windows, comprising:
for the entire time series, calculating mean, median, standard deviation, and variance for the acceleration vector magnitude associated with several time-window analyses of the data;
for each time-window, calculating the maximum, minimum, mean, median, standard deviation, and variance for the acceleration vector magnitude associated with each subset in a time progression of subsets subsumed within the entire time series of data;
for each subset, calculating the maximum, minimum, mean, median, standard deviation, and variance for the acceleration vector magnitude associated with the subset; and
calculating mean, median, standard deviation, and variance for the volume of the ellipsoid for the time series.

16. The method of claim 15, in which the summary statistics are selected from the group consisting of maximum, minimum, mean, median, standard deviation and variance.

17. The method of claim 9, in which the inertial motion sensing and reporting unit is attached to the subject near the subject's center of mass.

18. The method of claim 9 in which in step (xv) of calculating the visual adaptability to change statistic the selected motor tasks are (tandem stance, eyes open, firm surface compared to two legs, eyes open, firm surface), (tandem stance, eyes open, foam pad compared to two legs, eyes open, foam pad), (two legs, eyes open, foam pad compared to two legs, eyes open, firm surface), and (tandem stance, eyes open, foam pad compared to tandem stance, eyes open, firm surface).

19. The method of claim 9 in which in step (xvi) of calculating a vestibular adaptability to change statistic the selected motor tasks are (two legs, eyes closed, firm surface compared to two legs, eyes open, firm surface), (two legs, eyes closed, foam pad compared to two legs, eyes open, foam pad), (two legs, eyes open, foam pad compared to two legs, eyes open, firm surface), and (two legs, eyes closed, foam pad compared to two legs, eyes closed, firm surface).

20. The method of claim 9 in which in step (xvii) of calculating a somatosensory adaptability to change statistic the selected motor tasks are (two legs, eyes closed, firm surface compared to two legs, eyes open, firm surface), (tandem stance, eyes closed, firm surface compared to tandem stance, eyes open, firm surface), (tandem stance, eyes open, firm surface compared to two legs, eyes open, firm surface), and (tandem stance, eyes closed, firm surface compared to two legs, eyes closed, firm surface).

21. The method of claim 9 in which in step (xviii) of calculating a vision and vestibular integrated adaptability to change statistic the selected motor tasks are for (two legs, eyes open, foam pad compared to two legs, eyes open, firm surface), (two legs, eyes closed, foam pad compared to two legs, eyes closed, firm surface), (tandem stance, eyes open, foam pad compared to tandem stance, eyes open, firm surface), and (tandem stance, eyes closed, foam pad compared to tandem stance, eyes closed, firm surface).

22. The method of claim 9 in which in step (xix) of calculating a vision and somatosensory integrated adaptability to change statistic the selected motor tasks are (tandem stance, eyes open, firm surface compared to two legs, eyes open, firm surface), (tandem stance, eyes closed, firm surface compared to two legs, eyes closed, firm surface), (tandem stance, eyes open, foam pad compared to two legs, eyes open, foam pad), and (tandem stance, eyes closed, foam pad compared to two legs, eyes open, foam pad).

23. The method of claim 9 in which in step (xx) of calculating a vestibular and somatosensory integrated adaptability to change statistic the selected motor tasks are (two legs, eyes closed, firm surface compared to two legs, eyes open, firm surface), (tandem stance, eyes closed, firm surface compared to tandem stance, eyes open, firm surface), (two legs, eyes closed, foam pad compared to two legs, eyes open, foam pad), and (tandem stance, eyes closed, foam pad compared to tandem stance, eyes open, foam pad).

24. The method of claim 1, in which the at least one dual-task test of step (d) comprises at least one cognitive test component evaluating the subject's cognitive abilities administered contemporaneously with at least one postural stability testing component challenging the subject's postural stability.

25. The method of claim 24, in which the cognitive test component is selected from the group consisting of derivations of the trail-making test, parts A and B; the Wechsler adult intelligence scale digit span test, forward and backward; and the Stroop task.

26. The method of claim 24, in which the postural stability component is the tandem stance, eyes open test.

27. The method of claim 24, in which the Mi integrated performance score is produced by the steps of:
i) determining a peer group for the subject based on characteristics of the subject;
ii) administering a dual-task test to the subject;
iii) collecting data from an inertial motion sensing and reporting unit worn by the subject during the dual-task test;
iv) processing the data from the inertial motion sensing and reporting unit to produce subject processed data, and storing the subject processed data in a database;
v) calculating indicative dual-task statistics from the subject processed data;
vi) retrieving peer group statistics for the dual-task test as administered to the peer group from a database;
vii) calculating a peer group amplitude measure and frequency measure, comprising mean and standard deviation values for the peer group dual-task statistics;
viii) assigning an ordinal value to the mean and standard deviation values for the peer group dual-task statistics;
ix) based on mean and standard deviation values for the peer group dual-task statistics relative to the indicative dual-task statistics, calculating a dual-task score for the subject;
x) assigning an ordinal value to the dual-task score for the subject;
xi) calculating a single-task to dual-task change score by comparing the dual-task score for the subject to the score earned by the subject when performing the postural stability component of the dual-task test in step (d);
xii) calculating the Mi integrated performance score from a weighted average of values from steps (ii) through (xi).

28. The method of claim 24, in which the step (v) of calculating indicative dual-task statistics comprises:
A) for each time sample within a time series associated with each motor task, calculate an acceleration vector magnitude from components of linear acceleration along X, Y and Z axes;
B) for each time series associated with each motor task:
1) calculate summary statistics from the acceleration vector magnitudes for each of the time samples in the motor task;
2) perform a fast Fourier transform on each time series of the an acceleration vector magnitude and the components of linear acceleration along X, Y and Z axes, to determine a spectral centroid for each of the acceleration vector magnitude and the components of linear acceleration along X, Y and Z axes;
3) calculate volumetric statistics comprising the volume of an ellipsoid where the radii are the standard deviations of each of the components of linear acceleration along X, Y and Z axes;
4) for the entire time series, calculate time-window analysis statistics for a plurality of time windows, comprising:
for the entire time series, calculating mean, median, standard deviation, and variance for the acceleration vector magnitude associated with several time-window analyses of the data;
for each time-window, calculating the maximum, minimum, mean, median, standard deviation, and variance for the acceleration vector magnitude associated with each subset in a time progression of subsets subsumed within the entire time series of data;
for each subset, calculating the maximum, minimum, mean, median, standard deviation, and variance for the acceleration vector magnitude associated with the subset; and
calculating mean, median, standard deviation, and variance for the volume of the ellipsoid for the time series.

29. The method of claim 24, in which the summary statistics are selected from the group consisting of maximum, minimum, mean, median, standard deviation and variance.

30. The method of claim 1, in which the Mi evaluation summary report is displayed on a graph having a vertical axis and a horizontal axis meeting at a center point, in which four score axes are formed by the portions of the vertical axis above and below the center point and by the portions of the horizontal axis left and right of the center point, and in which the Mi evaluation summary is created by the steps of:
a) graphing the Mi symptoms summative score as a point along a first score axis;
b) graphing the Mi thinking composite score as a point along a second score axis;
c) graphing a value of the Mi balance composite stability score as a point along a third score axis;
d) graphing a value of the Mi integrated performance score as a point along a fourth score axis; and
e) connecting the points from steps (a), (b), (c) and (d) to form a four-sided, diamond-shaped graph.

31. The method of claim 30, in which in the center point represents a score of zero.

32. The method of claim 1, further comprising the step of storing the Mi evaluation summary report with a time of administration in a database.

33. The method of claim 32, further comprising the steps of:
a) retrieving at least one past Mi evaluation summary report with its time of administration from a database;
b) comparing the Mi evaluation summary report with the at least one past Mi evaluation summary report to detect changes in the subject over time.
